(19) United States
(12) Patent Application Publication
(10) Pub. No.: US 2025/0314663 A1
(43) Pub. Date: Oct. 9, 2025

(54) METHODS FOR TREATING ACUTE KIDNEY INJURY

(71) Applicant: Children's Hospital Medical Center, Cincinnati, OH (US)

(72) Inventors: Matthew Alder, Cincinnati, OH (US); Denise Hasson, Cincinnati, OH (US); Stuart Goldstein, Cincinnati, OH (US); Prasad Devarajan, Cincinnati, OH (US)

(73) Assignee: Children's Hospital Medical Center, Cincinnati, OH (US)

(21) Appl. No.: 18/866,194

(22) PCT Filed: May 19, 2023
(86) PCT No.: PCT/US2023/022837

§ 371 Date: Nov. 15, 2024

Related U.S. Application Data
(60) Provisional application No. 63/343,649, filed on May 19, 2022.
(60) Provisional application No. 63/462,844, filed on Apr. 28, 2023.

(51) Int. Cl. G01N 33/68; A61M 1/14

(52) U.S. Cl. CPC G01N 33/6893; A61M 1/14; G01N 2333/705; G01N 2800/347; G01N 2800/52

(57) ABSTRACT

The present invention provides methods relating to the discovery of olfactomedin 4 (OLFM4) as a biomarker for acute kidney injury (AKI) and need for renal replacement therapy, and further as a biomarker for responsiveness to the furosemide stress test (FST). The methods described here are useful in clinical decision support and personalized therapy for AKI, as well as for clinical trial design.

### CROSS-REFERENCE TO RELATED APPLICATIONS

This application claims priority from U.S. provisional application Ser. Nos. 63/343,649 filed May 19, 2022 and 63/462,844 filed Apr. 28, 2023, the contents of which are incorporated herein by reference in their entirety.

### STATEMENT REGARDING FEDERALLY SPONSORED RESEARCH

This invention was made with government support under K08GM124298 awarded by the National Institutes of Health. The government has certain rights in the invention.

### FIELD OF THE INVENTION

The invention disclosed herein generally relates to the use of olfactomedin 4 as a therapeutic biomarker, including in methods for treating patients having acute kidney injury, in methods for identifying patients in need of renal replacement therapy, and as a biomarker for responsiveness to the furosemide stress test.

### BACKGROUND

Acute kidney injury (AKI) occurs frequently in critically ill adults and children, and is associated with greater hospital costs, longer durations of high-risk interventions, greater intensive care unit (ICU) and hospital lengths of stay (LOS), and mortality. See Kaddourah et al. for a recent discussion of the epidemiology of AKI in critically ill children and young adults, NEJM 2017; 376:11-20. It is recognized that not all AKI is the same. Different etiologies, severity, duration, and timing of AKI can impact patient-specific outcomes. See Gist et al. for a discussion of transient and persistent acute kidney injury phenotypes following the Norwood operation in Cardiology in the Young 2021; 1-8; and Basu et al. for a discussion of clinical phenotypes of AKI associated with unique outcomes in critically ill septic children in Pediatric Research 2021; 90: 1031-1038.

AKI biomarkers have the potential to disentangle the clinical heterogeneity and help clinicians better understand the pathophysiology of AKI. The 23^{rd} Acute Disease Quality Initiative (ADQI) conference focused on AKI diagnostics and emphasized the importance of finding biomarkers that will refine AKI diagnosis based on pathophysiologic process, etiology, and location of injury (ADQI 23 2). See Ostermann et al. JAMA network open 2020; 3:e2019209. Of the 23 different biomarkers reviewed, none were expressed specifically by the loop of Henle (LOH). In addition to protein biomarkers, specific tests of renal tubular health can predict poor AKI outcomes. One of these, the furosemide stress test, can predict progression to stage III AKI (Chawla et al. Development and standardization of a furosemide stress test to predict the severity of acute kidney injury. Critical Care (London, England) 2013; 17: R207) and future receipt of renal replacement therapy (RRT) better than any of the new biomarker in adults or children. See discussion in Koyner J et al. Furosemide Stress Test and Biomarkers for the Prediction of AKI Severity. J. Am. Soc. Nephrol (JASN) 2015; 26: 2023-2031; Lumlertgul et al. Early versus standard initiation of renal replacement therapy in furosemide stress test non-responsive acute kidney injury patients (the FST trial). Critical Care (London, England) 2018; 22: 101; Kakajiwala et al. Lack of Furosemide Responsiveness Predicts Acute Kidney Injury in Infants After Cardiac Surgery. Annal. Thoracic Surg. 2017; 104: 1388-1394; and Gist et al. Urine Quantification Following Furosemide for Severe Acute Kidney Injury Prediction in Critically Ill Children. J Pediatr Intensive Care 2022;01: 1-82.

The present invention addresses the need for additional biomarkers in AKI to identify patients who are at risk of disease progression and therefore likely to benefit from early renal replacement therapy (RRT).

### SUMMARY

Provided are methods for treating acute kidney injury (AKI) in a human subject in need thereof. the methods comprising determining the amount of olfactomedin 4 (OLFM4) in a biological sample of the subject and administering renal replacement therapy (RRT) to the subject having an OLFM4 level above a pre-determined threshold value or administering supportive care to the subject having an OLFM4 level below the pre-determined threshold value.

Also provided are methods for determining whether a human subject in need thereof is likely to be responsive or unresponsive to furosemide administration, the methods comprising determining the amount of olfactomedin 4 (OLFM4) in a biological sample of the subject, wherein an OLFM4 level above a pre-determined threshold value indicates the subject is likely to be unresponsive and an OLFM4 level below the pre-determined threshold value indicates the subject is likely be furosemide responsive.

Also provided are methods for determining whether a human subject in need thereof is likely to progress to severe AKI, the methods comprising determining the amount of olfactomedin 4 (OLFM4) in a biological sample of the subject, wherein an OLFM4 level above a pre-determined threshold value indicates disease progression is likely and an OLFM4 level below the pre-determined threshold value indicates disease progression is less likely.

In one aspect, provided is a use of urinary olfactomedin 4 (OLFM4) in a method for treating acute kidney injury (AKI) in a human subject, the method includes determining an amount of olfactomedin 4 (OLFM4) in a urine sample of the subject; optionally where the method also includes determining an amount of neutrophil gelatinase-associated lipocalin (NGAL) in the urine sample.

In one aspect, provided is a use of urinary olfactomedin 4 (OLFM4) in a method for predicting furosemide responsiveness in a human subject, the method includes determining an amount of olfactomedin 4 (OLFM4) in a urine sample of the subject; optionally where the method also includes determining an amount of neutrophil gelatinase-associated lipocalin (NGAL) in the urine sample.

In one aspect, provided is a use of urinary olfactomedin 4 (OLFM4) in a method for identifying a subject in need of renal replacement therapy (RRT), the method includes determining an amount of olfactomedin 4 (OLFM4) in a urine sample of the subject; optionally where the method also includes determining an amount of neutrophil gelatinase-associated lipocalin (NGAL) in the urine sample. In aspects of the disclosure, the method may include determining a product of the urine OLFM4 and urine NGAL levels in the sample.

In accordance with any of the methods described here, the subject in need may be a subject diagnosed with stage 1 or stage 2 AKI, optionally using the Kidney Disease: Improving Global Outcomes (KDIGO) scale.

In accordance with any of the methods described here, the subject in need may be a subject diagnosed with stage 0-1 or stage 2-3 AKI, optionally using the Kidney Disease: Improving Global Outcomes (KDIGO) scale.

In accordance with any of the methods described here, the subject in need may be one predicted to have kidney injury based on analysis of the subject's electronic medical record and/or one having urinary NGAL levels greater than 100 ng/ml, greater than 150 ng/ml, or greater than 200 ng/ml.

In some aspects of the disclosure, the subject in need may be a subject who is hemodynamically unstable and/or a subject who is hypervolemic or hypovolemic.

In some aspects of the disclosure, the subject in need may be a subject who is hemodynamically stable.

In some aspects of the methods described here, the subject has not been administered furosemide or received a furosemide stress test (FST) prior to determining the amount of OLFM4 in the biological sample.

In aspects of the disclosure, the biological sample is a urine sample.

In aspects of the disclosure, supportive care comprises one or more of fluid management, maintenance of euvolemia, prevention of hypotension, and avoidance of nephrotoxins.

In aspects of the disclosure, the method further comprises monitoring serum creatinine and urine output, optionally wherein the method further comprising detecting serum creatinine levels in one or more additional biological samples of the patient obtained at times following the initial determination of OLFM4.

In accordance with any of the methods described here, the method may comprise assaying serum and/or urine neutrophil gelatinase-associated lipocalin (NGAL) levels in a biological sample of the patient.

In aspects of the disclosure, the pre-determined threshold value of OLFM4 is 30 nanograms per milliliter (ng/mL), 40 ng/ml, 50 ng/ml, 100 ng/ml, 150 ng/ml, 200 ng/ml, 250 ng/ml, 300 ng/ml, or 350 ng/ml.

In aspects of the disclosure, the pre-determined threshold value of OLFM4 is 300 ng/ml, 350 ng/ml, 400 ng/ml, 450 ng/ml, 500 ng/ml, or 550 ng/ml in urine.

In aspects of the disclosure, the method may comprise determining an amount of uromodulin in a biological sample of the subject and calculating a ratio of uromodulin to OLFM4, wherein a ratio below a pre-determined threshold value indicates the subject is likely to progress to a more severe form of AKI, require RRT, and/or fail to respond to furosemide. In aspects of the disclosure, the ratio is from 0.1-4, or wherein the ration is selected from 4, 2, 1, 0.5, 0.25, and 0.1.

In some aspects, the subject is diagnosed with sepsis.

In other aspects, the subject is not septic.

In aspects of the disclosure, determining the amount of olfactomedin 4 (OLFM4) in the biological sample comprises subjecting a portion of the sample to an immunoassay utilizing an anti-OLFM4 antibody.

### BRIEF DESCRIPTION OF THE DRAWINGS

FIG. 1A Box and whisker plot showing urine OLFM4 levels from patients in 4 groups: (1) no sepsis and no AKI (n=8), (2) sepsis and no AKI (n=10), (3) AKI and no sepsis (n=7), or (4) septic AKI (n=11), p=0.0342. **p<0.005.

FIG. 1B Box and whisker plot showing urine NGAL levels from patients in the same 4 groups as FIG. 1A (n=8, 10, 6, and 11 respectively), p=0.0038. **p<0.005.

FIG. 1C Box and whisker plot showing urine olfactomedin 4 (uOLFM4) levels in patients with no/stage 1 AKI versus stage 2-3 severe AKI (n=17-18/group), p=0.0435 and 0.0027, respectively. *p<0.05.

FIG. 1D Box and whisker plot showing urine neutrophil gelatinase associated lipocalin (uNGAL) levels in patients with no/stage 1 AKI versus stage 2-3 severe AKI (n=17-18/group), p=0.0435 and 0.0027, respectively. **p<0.005.

FIG. 2A Box and whisker plot showing urine OLFM4 levels in patients without versus with sepsis (n=14-15 and 21, respectively), p=0.0255. *p<0.05.

FIG. 2B Box and whisker plot showing urine NGAL levels in patients without versus with sepsis (n=14-15 and 21, respectively), p=0.0255 and p=0.0095. **p<0.005.

FIG. 2C Box and whisker plot showing urine OLFM4 levels in septic patients with no AKI versus with septic AKI (n=10 and 11, respectively), p=0.1321.

FIG. 2D Box and whisker plot showing urine NGAL levels in septic patients with no AKI versus with septic AKI (n=10 and 11, respectively), p=0.0027. **p<0.005.

FIG. 3A Spearman correlation between uOLFM4 and uNGAL levels by individual patient.

FIG. 3B Individual patient levels of uOLFM4 (triangles) and uNGAL (boxes), grouped by AKI and sepsis status. Two patients had uNGAL levels >5000, shown at the limit of the y-axis. Asterisk (*) indicates patients with disparate uOLFM4 and uNGAL values.

FIG. 4A Receiver operating curve showing ability of urine OLFM4 levels to discriminate between patients without AKI versus with AKI.

FIG. 4B Receiver operating curve showing ability of urine NGAL levels to discriminate between patients without AKI versus with AKI.

FIG. 4C Receiver operating curve showing ability of the product of both urine OLFM4 levels and urine NGAL levels to discriminate between patients AKI versus with AKI.

FIG. 5: Immunofluorescence from 3 human biopsy samples with acute tubular necrosis. Columns left to right show background, OLFM4, uromodulin, and merged OLFM4 and uromodulin images. Background shows distorted tubular architecture from AKI. OLFM4 staining appears in white. Uromodulin, staining loop of Henle cells, appears in red. Merged images of the white OLFM4 overlaying the red loop of Henle cells. Controls non-AKI samples had very rare OLFM4 staining or were devoid of OLFM4 altogether. ATN-acute tubular necrosis. OLFM4-olfactomedin4.

FIG. 6A Box and whisker plot showing uOLFM4 levels in patients with and without sepsis and AKI. Linear mixed modeling was used for testing on log transformed data. For each of the groups, n=33, 15, 67, and 63, in the same order as presented in the figure. Asterisk (*) represents p<0.001.

FIG. 6B Box and whisker plot showing uNGAL levels in patients in the same four groups as FIG. 6A. For each of the groups, n=33, 14. 67, and 63, in the same order as presented in the figure. Asterisk (*) represents p<0.001.

FIG. 7A Box and whisker plot showing pairwise comparisons of uOLFM4 levels in all patients with and without AKI. n=48, 130; p<0.001. Linear mixed modeling was used for testing on log transformed data for all pairwise comparisons.

FIG. 7B Box and whisker plot showing pairwise comparisons of uNGAL levels in all patients with and without AKI. n=48, 130, p<0.001.

FIG. 7C Box and whisker plot showing pairwise comparisons of uOLFM4 in only the no AKI subset of patients, comparing patients without and with sepsis. n=33, 15; p=0.69.

FIG. 7D Box and whisker plot showing pairwise comparisons of uNGAL in only the no AKI subset of patients, comparing patients without and with sepsis. n=33, 14; p=0.18.

FIG. 7E Box and whisker plot showing pairwise comparisons of uOLFM4 in only the sepsis subset of patients, comparing patients without and with AKI. n=15, 63; p=<0.001.

FIG. 7F Box and whisker plot showing pairwise comparisons of uNGAL in only the sepsis subset of patients, comparing patients without and with AKI. n=14, 63; p=0.006.

FIG. 8A Box and whisker plot showing uOLFM4 is higher in those patients who failed to respond to furosemide compared to those who made adequate urine following furosemide administration. n=19, 27; p=0.04. Linear mixed modeling was used for testing on log transformed data used for pairwise comparisons.

FIG. 8B Box and whisker plot showing uNGAL is not different when comparing those patients who failed FST compared to those who made adequate urine following FST. n=19, 26; p=0.11.

FIG. 8C Receiver operating curve showing uOLFM4 ability to predict furosemide responsiveness: AUC=0.751 (0.60-0.89); p=0.0041.

FIG. 9A Box and whisker plot showing uromodulin alone is not different between patients with and without AKI. n=25, 76; p=0.16. For these comparisons, log transformed data was used with a Mann-Whitney test rather than linear mixed model due to smaller numbers.

FIG. 9B Box and whisker plot showing uromodulin:OLFM4 ratio is lower in those patients with AKI. n=25, 76; p =<0.0001. These results show that uromodulin:OLFM4 ratio predicts AKI.

FIG. 9C Box and whisker plot showing uromodulin:OLFM4 ratio is also lower in those patients who failed to respond to furosemide. n=11, 12; p=0.044. These results show that uromodulin:OLFM4 ratio failure to respond to furosemide.

FIG. 10A Box and whisker plot showing uOLFM4 is elevated in severe AKI and patients who required RRT. uOLFM4 levels in three groups are shown. Group 1 (0-1) is AKI staging using KDIGO definitions, thus group one is no kidney injury to mild kidney injury. Group 2 (2-3) represents severe kidney injury. Group 3 (RRT) represents those patients who needed renal replacement therapy. Number of patients in each group is represented by n=with the numbers in the same order as the patient groups. Bars with p values demonstrate statistical differences between groups using ANOVA-Kruskal Wallis to assess for differences between groups.

FIG. 10B Box and whisker plot showing uNGAL is elevated with AKI. uNGAL levels in three groups are shown. Group 1 (0-1) is AKI staging using KDIGO definitions, thus group one is no kidney injury to mild kidney injury. Group 2 (2-3) represents severe kidney injury. Group 3 (RRT) represents those patients who needed renal replacement therapy. Number of patients in each group is represented n=with the numbers in the same order as the patient groups. Bars with p values demonstrate statistical differences between groups using ANOVA-Kruskal Wallis to assess for differences between groups.

FIG. 11A Box and whisker plot showing uOLFM4 is elevated in patients who require RRT (RRT) versus those who do not (No RRT). This is comparing first uOLFM4 levels after RAI trigger (mostly 12 hours after admission) in those patients who required renal replacement therapy (RRT) and those who did not. Number and bar graph depict p values for Mann Whitney test. Number of patients in each group is shown below n=with the numbers in the same order as the groups.

FIG. 11B Box and whisker plot showing uNGAL levels in patients who require RRT (RRT) versus those who did not (No RRT). This is comparing first uOLFM4 levels after RAI trigger (mostly 12 hours after admission) in those patients who required renal replacement therapy (RRT) and those who did not. Number and bar graph depict p values for Mann Whitney test. Number of patients in each group is shown below n=with the numbers in the same order as the groups.

FIG. 12A Box and whisker plot showing uOLFM4 is elevated in patients who fail to respond to furosemide (FST Neg) versus those who respond (FST Pos). This is comparing first uOLFM4 levels after RAI trigger (mostly 12 hours after admission) in those patients who responded to furosemide and those who did not. Number and bar graph depict p values for Mann Whitney test. Number of patients in each group is shown below n=with the numbers in the same order as the groups.

FIG. 12B Box and whisker plot showing uNGAL levels in patients who fail to respond to furosemide (FST Neg) versus those who respond (FST Pos). This is comparing first uOLFM4 levels after RAI trigger (mostly 12 hours after admission) in those patients who responded to furosemide and those who did not. Number and bar graph depict p values for Mann Whitney test. Number of patients in each group is shown below n=with the numbers in the same order as the groups.

### DETAILED DESCRIPTION OF THE INVENTION

The present disclosure provides olfactomedin 4 (OLFM4) as a biomarker for acute kidney injury (AKI) and related methods, including methods for predicting furosemide responsiveness in the furosemide stress test (FST) and for identifying AKI patients at low or high risk of progression to severe AKI, including patients likely to require renal replacement therapy (RRT). As discussed in more detail below, recent studies have demonstrated that FST responsive AKI patients rarely progress to severe disease requiring RRT. However, a significant limitation of the FST is the need for the patient to be hemodynamically stable and euvolemic for this test to be administered. The methods described here predict furosemide responsiveness without the need for hemodynamic stability. Accordingly, the present methods provide an alternative means to identify patients who are likely to progress to severe disease requiring RRT. Administration of RRT early in the course of disease progression, before injury becomes severe or irreversible, is one means to improve patient outcomes in AKI. Accordingly, in one aspect, the invention provides methods of treating AKI by identifying patients at high risk of disease progression for early administration of RRT.

Accordingly, the methods described here are useful in clinical decision support, including point-of-care (“POC”) clinical decision making based on the needs of the individual patient. The methods are useful to identify patients likely to progress to severe AKI, which cohort of patients is also likely to benefit from more aggressive therapy, such as RRT, as opposed to less aggressive forms of supportive care, including fluid management, maintenance of euvolemia, prevention of hypotension, and avoidance of nephrotoxins as able. Thus, the methods are also useful for identifying patients who will likely recover without progression to severe AKI and therefore are useful to reduce exposure to aggressive interventions such as RRT in low-risk patients. The identification of high and low risk patient cohorts using the methods described here can also be incorporated into methods for clinical trial design.

AKI is defined in accordance with clinical practice. See for example the 2012 Kidney Disease Improving Global Outcomes (KDIGO) Clinical Practice Guidance. See Kellum et al., Kidney International Supplements 2012 2:1-138 in J. Int'l Society of Nephrology 2(1) March 2012, Suppl. 1. In some aspects of the methods described here, a subject having AKI or a subject diagnosed with AKI is one who has been diagnosed with AKI in accordance with any one of the following criteria (i) increase in serum creatine (SCr) by ≥0.3 mg/dl (≥26.5 μmol/l) within 48 hours; (ii) increase in SCr to ≥1.5 times baseline, which is known or presumed to have occurred within the prior 7 days; or (iii) a urine volume<0.5 ml/kg/h for 6 hours.

In the context of the present invention, the term “early AKI” or “early-stage AKI” refers to stage 1 AKI as determined by the KDIGO stage for acute kidney injury (AKI), or “KIDGO AKI stage”. The term “moderate AKI” refers to KIDGO stage 2, and the term “severe AKI” refers to KIDGO stage 3. The KIDGO definitions and staging of AKI are based on the Risk, Injury, Failure; Loss, End-Stage Renal Disease (RIFLE) and Acute Kidney Injury Network (AKIN) criteria and studies on risk relationship. The 2012 KIDGO AKI stages are described in Table 1 for purposes of illustration. It is understood that the claimed methods may be practiced in accordance with alternative, but similar guidance available to the skilled person, such as the Acute Kidney Injury Network (AKIN) stages. which are also based on RIFLE.

Based on the KDIGO criteria, AKI is staged into three stages of increasing severity based upon serum creatinine levels and urine output. Accordingly, in some aspects, the methods described here may further comprise determining or receiving additional clinical data of the subject, such as the subject's serum creatinine levels and urine output.

TABLE 1
KIDGO Stages (adapted from Kellum et al. 2012)

| Stage | Serum creatinine | urine output |
|---|---|---|
| 1 | 1.5-1.9 times baseline OR ≥0.3 | <0.5 ml/kg/h for 6-12 hrs |
| | mg/dl | |
| 2 | 2.0-2.9 times baseline | <0.5 ml/kg/h for ≥12 hrs |
| 3 | 3.0 times baseline OR ≥4.0 | <0.3 ml/kg/h for ≥24 hrs OR |
| | mg/dl | anuria for ≥12 hours |

In some of the methods described here, the subject is one diagnosed with AKI and/or one presenting with KIDGO stage 1 or KIDGO stage 2 AKI. In some aspects of the methods described here, a subject in need of therapy for AKI is one presenting with KIDGO stage 1 or KIDGO stage 2 AKI, or one presenting with KIDGO stage 3 AKI who has not yet received RRT.

In aspects of the disclosure, the subject is further defined as one who is hemodynamically unstable and/or not euvolemic. In some aspects, the hemodynamically unstable subject is one whose blood pressure is abnormal or unstable and/or whose heart rate is abnormal, for example a heart rate characterized by arrhythmias or characterized as a higher rate than is expected based on chronological age. In some aspects, the subject may be hypervolemic or hypovolemic.

In some aspects, the subject is hemodynamically stable.

In some of the methods described here, a subject in need of therapy for AKI is one having AKI resulting from sepsis, critical illness, circulatory shock, burns, trauma, cardiac surgery, major noncardiac surgery, nephrotoxic drugs, radiocontrast agents, and poisonous animals or plants. In some aspects, the subject in need of therapy for AKI may further be characterized as having one or more susceptibilities to AKI selected from dehydration or volume depletion, advanced age, female gender, black race, chronic disease of the heart, lung, or liver, diabetes mellitus, cancer, and anemia. Accordingly, the methods described here may further incorporate patient specific clinical data including one or more of the foregoing co-morbidities and/or patient demographical information.

In some aspects of the disclosure, the detection of OLFM4 described here may be used in combination with additional patient specific biomarker data. In some aspects, the methods further comprise receiving patient specific biomarker data including creatinine levels and urine output. In some aspects, the methods further comprise receiving patient specific biomarker data for one or more additional biomarkers selected from uromodulin, plasma neutrophil gelatinase-associated lipocalin (NGAL), urinary IL-18, tissue inhibitor of metalloproteinases (TIMP-2) and IGF-binding protein-7 (IGFBP-7). In some aspects, the methods further comprise detecting one or more additional patient specific biomarkers selected from uromodulin, NGAL, IL-18, TIMP-2 and IGFBP-7. In some aspects, the invention provides a companion diagnostic for AKI progression that may be used in combination with one or more additional patient specific biomarkers selected from uromodulin, NGAL, IL-18, TIMP-2 and IGFBP-7.

Unless otherwise noted, terms are to be understood according to conventional usage by those of ordinary skill in the relevant art.

As used herein, the term “subject” refers to a mammal, for example a mouse, a rat, a dog, a guinea pig, a non-human primate, or a human. In some aspects, the subject is a human. The term “patient” refers to a human subject. In some aspects, the subject is a pediatric patient. A pediatric patient is defined as one under 18 years of age.

As used herein, the terms “treatment,” “treating,” “treat,” and the like, refer to obtaining a desired pharmacologic and/or physiologic effect in relation to a disease or disorder. The effect is therapeutic in terms of achieving a clinical response, which may be partial or complete, and may alleviate one or more symptoms attributable to the disease or disorder being treated.

As used herein, the term “marker” or “biomarker” refers to a biological molecule, such as, for example, a nucleic acid, a peptide, a protein, or a small biomolecule such as a creatinine, whose presence or absence, or concentration in a biological sample, can be detected and correlated with a clinical diagnosis, a clinical prognosis, or a clinical risk. In some aspects, the biomarker is a protein or peptide detectable in the urine, serum or blood of a mammalian subject. In some aspects, the methods described here further comprise detecting serum creatinine levels in a biological sample obtained from a subject. In some aspects of the methods described here, urine output may also constitute a biomarker.

### Olfactomedin 4 (OLFM4)

Olfactomedin 4 (OLFM4) is a secreted glycoprotein expressed in mature neutrophils and epithelial cells in prostate and gut epithelium following stress. In states of normal health, only about ˜25% of human neutrophils express OLFM4 (Clemmensen et al. Olfactomedin 4 defines a subset of human neutrophils. J. Leuk. Biol. 2012; 91: 495-500); however, it is one of the most upregulated genes in the peripheral blood of patients with sepsis (Wong et al. Genomic expression profiling across the pediatric systemic inflammatory response syndrome, sepsis, and septic shock spectrum. Crit. Care Med. 2009; 37: 1558-1566). In pediatric patients with septic shock, increased OLFM4 mRNA transcription, plasma protein levels, and a greater percentage of OLFM4 positive neutrophils are independently associated with multiorgan failure and death (Alder et al. Olfactomedin 4 marks a subset of neutrophils in mice. Innate Immunity 2019; 25: 22-33). In addition, OLFM4 null mice are protected from death in sepsis models, suggesting its role in the immune response. See Liu W et al. Olfm4 deletion enhances defense against Staphylococcus aureus in chronic granulomatous disease. J. Clin. Invest. 2013; 123: 3751-3755; and Liu W et al. Olfactomedin 4 down-regulates innate immunity against Helicobacter pylori infection. PNAS USA 2010; 107: 11056-11061.

Wild type murine pups challenged with sepsis showed increased OLFM4 expression that localized to the kidney, specifically to the loop of Henle (LOH). Healthy control animals do not express OLFM4; only following septic challenge and renal injury was OLFM4 expression detected in the LOH and in the urine of mice. See Stark JE, et al. Juvenile OLFM4-null mice are protected from sepsis. Am. J. Physiol. Renal Physiol. 2020; 318: F809-F816.

Example 1 infra describes a retrospective pilot study undertaken to test whether OLFM4 could be detected in the urine of human AKI patients and, if detected, whether it was increased in patients with AKI and sepsis. The data in Example 1 demonstrate that OLFM4 was detectable in human urine, that it was elevated in patients with AKI and sepsis, and further that OLFM4 localizes to the LOH in human patients. Urinary OLFM4 protein correlates with creatinine-diagnosed AKI, providing an LOH specific biomarker for AKI in humans.

In Example 2, the main findings of Example 1 were validated in a larger prospective cohort of critically ill pediatric patients. In addition, the data in Example 2 extend these findings to show that OLFM4 can identify kidney injury and predict furosemide responsiveness.

In Example 3, the main findings of Example 1 were further validated in another cohort of critically ill pediatric patients.

As discussed in more detail below, urinary OLFM4 (uOLFM4) can identify patients having severe AKI and predict failure to respond to furosemide in AKI patients. Accordingly, provided is a new biomarker for AKI disease progression useful for identifying patients in need of aggressive therapy such as RRT prior to onset of severe AKI.

In accordance with some of the methods described here, the methods comprise determining the amount of olfactomedin 4 (OLFM4) in a biological sample of a subject in comparison to a pre-determined threshold value. The threshold value is the threshold for the state being measured by the assay and can be defined as a one-dimensional quantitative score, or “cut-off” value which refers to the diagnostic cut-off value, based upon receiver operating characteristic (ROC) analysis. ROC analysis is utilized to identify an optimal threshold value or diagnostic cut-off value (these terms are used synonymously herein), which is the value that optimizes the sensitivity and specificity of the test. For a detailed, non-mathematical discussion of ROC analysis, see Park et al., Korean J Radiol. 2004 January-Mar; 5(1):11-18. The following discussion is intended for illustrative purposes to provide context for the methods described herein.

The ROC curve is a plot of test sensitivity (y-axis) versus the inverse of test specificity, also referred to as the test false positive rate (FPR) (x-axis). This plot describes the inverse relationship between sensitivity and specificity across a series of cut-off values. Each discrete point on the graph is referred to as an operating point and is generated by using different cutoff levels for a positive test result. An ROC curve is estimated from these discrete points based on an assumption that the test results follow a certain distribution, e.g., a binormal distribution. The resulting curve is referred to as a fitted or smooth ROC curve. Estimation of the smooth ROC curve based on a binormal distribution utilizes maximum likelihood estimation (MLE). When a binormal distribution is used, the shape of the smooth ROC curve is determined by two parameters, a and b, which refer to (a) the standardized difference in the means of the distributions of the test results for those subjects with and without the condition and (b) the ratio of the standard deviations of the distributions of the test results for those subjects without versus those with the condition. It is also possible to construct an empirical ROC curve by connecting all the points obtained at all possible cutoff levels. Since the ROC curve describes the sensitivities and false positive rates at all possible cut-off values, it can be used to assess the performance of a test independently of a particular threshold value, since the area under the ROC curve (AUC) is a combined measure of a test's sensitivity and specificity. AUC is therefore indicative of the overall performance of a diagnostic test. AUC takes a value between 0 and 1. Values closer to 1 indicate better performance, with a value of 1 indicating perfect accuracy. In practice, the lower limit for the AUC of a clinical diagnostic test is 0.5. AUC values less than 0.5 indicate test performance worse than relying on chance. Preferably, to be useful clinically, the test should have an AUC of at least 0.70, or at least 0.75, or at least 0.80. AUC is often presented along with its 95% confidence interval (CI). The CI represents a range of values within which the true value of AUC can be found within the degree of confidence is selected. Thus the 95% CI is the range of values in which the true value lies within a 95% degree of confidence. In some aspects, the methods described here predict furosemide responsiveness or progressive disease with an AUC of at least 0.70, at least 0.75, or at least 0.80 with a 95% CI.

In accordance with the methods described here, the value chosen as the diagnostic threshold or cut-off value is one that optimizes performance of the test in the context of AKI. In this context, the value is chosen to obtain a high specificity and low false positive rate to minimize false positive decisions and consequently exposing patients unnecessarily to early aggressive therapies such as RRT. Thus, the value is selected to identify patients who are most likely to need RRT and may benefit from early initiation of RRT. In aspects of the disclosure, a concentration of urinary OLFM4 (uOLFM4) of 30 nanograms per milliliter (ng/ml), 40 ng/ml, 50 ng/ml, 100 ng/ml, 150 ng/ml, 200 ng/ml, 250 ng/ml, 300 ng/ml, or 350 ng/ml is selected as the pre-determined threshold value. In aspects of the disclosure, a concentration of urinary OLFM4 (uOLFM4) of 50 nanograms per milliliter (ng/ml) is selected as the pre-determined threshold value. In aspects of the disclosure, a concentration of urinary OLFM4 (uOLFM4) of 400 ng/ml, 450 ng/ml, 500 ng/ml, 550 ng/ml, 600 ng/ml, 650 ng/ml, 700 ng/ml, or 750 ng/ml is selected as the pre-determined threshold value. In aspects of the disclosure, a concentration of urinary OLFM4 (uOLFM4) of 500 ng/ml, 750 ng/ml, or 1000 ng/ml is selected as the pre-determined threshold value.

### Furosemide Stress Test (FST)

The furosemide stress test (FST) utilizes a standardized dose of the diuretic, furosemide, to test the function capacity of the of Loop of Henle (LOH) as well as the overall health of the renal tubular system. Chawla et al., Development and standardization of a furosemide stress test to predict the severity of acute kidney injury. Crit Care 17: R207, 2013. Specifically, using receiver-operating characteristic curve (AUC) analysis, Chawla showed that the area under the curve (AUC) for urine output 2 hours after furosemide administration to predict progression to AKI Network (AKIN) Stage-3 AKI in 77 patients (±SEM) was 0.87+0.09; P=0.001. Chawla reported the ideal cutoff for predicting progressive AKI during these first 2 hours was a urine volume less than 200 ml (100 ml/hr) with a sensitivity of 87.1% and a specificity of 84.1%.

A urine output of less than 3 ml/kg/hr in the 4 hours following furosemide administration in children is generally understood as predictive of progression to stage 3 AKI and future need for renal replacement therapy (RRT).

The FST has also outperformed several biomarkers for predicting progressive AKI and the need for RRT, including fractional excretion of sodium (FeNa), urine and plasma NGAL, urine albumin-to-creatinine ratio, urinary IL-18, kidney injury molecule-1 (KIM-1), TIMP2, IGFBP-7, and uromodulin. See Koyner et al., Furosemide Stress Test and Biomarkers for the Prediction of AKI Severity; JASN August 2015, 26 (8) 2023-2031.However, to perform the FST, the patient must be hemodynamically stable and euvolemic. Accordingly, the timing of the FST is often not optimal when patients are critically ill, however, these same patients stand to gain the most benefit from the information the FST provides, which may indicate, for example, the need for aggressive therapy such as RRT.

### Biological Sample Acquisition and Analysis

The methods described here may include obtaining or acquiring a biological sample from a subject. Preferably the sample is acquired in the 24 to 72 hours before a first administration of furosemide to the subject, to test furosemide responsiveness. In some aspects, a sample is acquired within the first 24 or 48 hours prior to furosemide administration. In some aspects, more than one sample may be acquired, for example in order to monitor any changes in furosemide responsiveness over a period of time.

The methods described here may include detecting and/or determining the amount or level (these terms are used interchangeably) of a biomarker in a biological sample obtained from a subject. In aspects of the disclosure, the biological sample is a urine, serum, plasma, or whole blood sample. In aspects of the disclosure, the biological sample is a urine sample.

In aspects of the disclosure, the biomarker is a protein or peptide biomarker. Protein and peptide-based biomarkers can be determined by methods known to the skilled person. For example, using a multiplex magnetic bead platform to isolate the analyte from the biological sample, such as those commercially available from the Millipore Corp (Billerica, MA) and known, for example, by the tradename MILLIPLEX™ MAP. Analyte concentration may be measured, for example, using a system such as that known by the tradename Luminex® (Luminex Corporation, Austin, TX), according to the manufacturers' specifications. These examples are not intended to be limiting, only illustrative. Any method or system for isolating, detecting or quantifying the amount of a protein or peptide-based biomarker in a biological sample can be used.

In aspects of the disclosure, the biomarker is detected by a method comprising one or more of electrophoresis, chromatography, immunoassay, or spectrometry, including mass spectrometry, fluorescence spectroscopy, infrared spectroscopy, and Raman spectroscopy. In some aspects, the method comprises high pressure liquid chromatography and mass spectroscopy (HPLC-MS). In some aspects, the method comprises an immunoassay selected from radioimmunoassay (RIA), enzyme immunoassay (EIA), enzyme-linked immunosorbent assay (ELISA), fluoroimmunoassay (FIA), chemiluminescence immunoassay (CLIA), liposome immunoassay (LIA) and capillary electrophoresis immunoassay (CEIA).

Immunoassay methods generally utilize analyte-specific antibodies, preferably monoclonal antibodies, a detectable signal-generating label, and a separation matrix. The detectable label may be selected from a radiolabel, e.g., 25I, 3H, and 14C, or more preferably a non-radioactive label such as an enzyme, a fluorescent molecule, a chemiluminescent substance, a metal or metal chelate, or a liposome. Suitable separation matrices permit separation of the immune complex formed during the assay, e.g., the complex formed from the binding of the analyte-specific antibody to analyte, and include charcoal, polyethylene glycol, a second antibody, microbeads, and microtiter plates, such as the 96-well plate. The methods may comprise a competitive immunoassay, e.g., antigen-capture or antibody-capture, or non-competitive immunoassay, e.g., “sandwich” ELISA.

The methods described here comprise detection of OLFM4 protein in a biological sample of a subject, preferably a urine sample. In aspects of the disclosure, OLFM4 protein is detected using an immunoassay comprising an anti-OLFM4 antibody. In aspects of the disclosure, the antibody is a human or humanized monoclonal anti-OLFM4 antibody. In aspects of the disclosure, the antibody is a polyclonal anti-OLFM4 antibody that reacts with human OLFM4. Suitable antibodies are commercially available, for example, from ThermoFisher Scientific (Invitrogen), R&D Systems, Millipore (Sigma), and Abcam.

In an exemplary aspect, urine OLFM4 is detected by immunoassay utilizing a fluorescent bead-based assay such as provided in the Luminex xMAP system. In another exemplary aspect, urine OLFM4 is detected by a method comprising an enzyme-linked immunosorbent assay (ELISA).

In some aspects of the disclosure, the methods may further comprise detecting and/or determining the amount of one or more additional biomarkers selected from uromodulin, plasma neutrophil gelatinase-associated lipocalin (NGAL), urinary interleukin 18 (IL-18), tissue inhibitor of metalloproteinases (TIMP-2) and insulin-like growth factor binding protein-7 (IGFBP-7). In some aspects of the disclosure, the methods comprise detecting one or more of serum uromodulin and urine or serum NGAL.

### Additional Patient Information

Demographic data, additional clinical characteristics, and/or results from other tests may impact prognosis. Accordingly, such demographic data, clinical characteristics, and/or results from other tests or indicia of AKI and/or co-morbidities may be incorporated into the methods described herein. In aspects of the disclosure, patient clinical characteristics include co-morbidities selected from sepsis, critical illness, circulatory shock, burns, trauma, cardiac surgery, major noncardiac surgery, nephrotoxic drugs, radiocontrast agents, and poisonous animals or plants. In aspects of the disclosure, the patient clinical characteristics may include susceptibility factors such as dehydration or volume depletion, advanced age, female gender, black race, chronic disease of the heart, lung, or liver, diabetes mellitus, cancer, and anemia. Accordingly, the methods described here may further incorporate patient specific clinical data including one or more of the foregoing co-morbidities and/or patient demographic information.

In aspects of the disclosure, patient demographic data includes one or more of the patient's age, race, and gender. In aspects of the disclosure, patient clinical characteristics include one or more of the patient's co-morbidities. In aspects of the disclosure, the co-morbidities may include sepsis, cancer, circulatory shock, burns, trauma, cardiac disease, cardiac surgery, major noncardiac surgery, nephrotoxic drugs, radiocontrast agents, and poisonous animals or plants. Other co-morbidities can include acute lymphocytic leukemia, acute myeloid leukemia, anemia, aplastic anemia, atrial and ventricular septal defects, bone marrow transplantation, caustic ingestion, chronic granulomatous disease, chronic hepatic failure, chronic lung disease, chronic lymphopenia, chronic obstructive pulmonary disease (COPD), congestive heart failure (NYHA Class IV CHF), Cri du Chat syndrome, cyclic neutropenia, developmental delay, diabetes, DiGeorge syndrome, Down syndrome, drowning, end stage renal disease, glycogen storage disease type 1, hematologic or metastatic solid organ malignancy, hemophagocytic lymphohistiocytosis, hepatoblastoma, heterotaxy, hydrocephalus, hypoplastic left heart syndrome, IPEX Syndrome, kidney transplant, Langerhans cell histiocytosis, liver and bowel transplant, liver failure, liver transplant, medulloblastoma, metaleukodystrophy, mitochondrial disorder, multiple congenital anomalies, multi-visceral transplant, nephrotic syndrome, neuroblastoma, neuromuscular disorder, obstructed pulmonary veins, Pallister Killian syndrome, Prader-Willi syndrome, requirement for chronic dialysis, requirement for chronic steroids, retinoblastoma, rhabdomyosarcoma, rhabdosarcoma, sarcoma, seizure disorder, severe combined immune deficiency, short gut syndrome, sickle cell disease, sleep apnea, small bowel transplant, subglottic stenosis, tracheal stenosis, traumatic brain injury, trisomy 18, type 1 diabetes mellitus, unspecified brain tumor, unspecified congenital heart disease, unspecified leukemia, VATER Syndrome, Wilms tumor, and the like. Any one or more of the above patient co-morbidities can be indicative of the presence or absence of chronic disease in the patient.

### Methods of Treating and Diagnosis

In aspects of the disclosure, the present invention provides methods of treating acute kidney injury (AKI) in a human subject in need thereof, the methods comprising determining the amount of olfactomedin 4 (OLFM4) in a biological sample of the subject and administering renal replacement therapy (RRT) to the subject having an OLFM4 level above a pre-determined threshold value or administering renal supportive care to the subject having an OLFM4 level below the pre-determined threshold value.

In aspects of the disclosure, the invention provides methods for determining whether a human subject in need thereof is likely to be responsive or unresponsive to furosemide administration, e.g., in a furosemide responsiveness test (FST), the method comprising determining the amount of olfactomedin 4 (OLFM4) in a biological sample of the subject, wherein an OLFM4 level above a pre-determined threshold value indicates the subject is likely to be unresponsive and an OLFM4 level below the pre-determined threshold value indicates the subject is likely be furosemide responsive.

In aspects of the disclosure, the invention provides methods for determining whether a human subject in need thereof is likely to progress to severe AKI, the method comprising determining the amount of olfactomedin 4 (OLFM4) in a biological sample of the subject, wherein an OLFM4 level above a pre-determined threshold value indicates disease progression is likely and an OLFM4 level below the pre-determined threshold value indicates disease progression is less likely.

In some aspects, the methods described here include treating the subject identified as at low risk of disease progression by excluding the low-risk subject from one or more aggressive and/or high-risk therapies, such as renal replacement therapy (RRT).

The methods and materials of the invention are expressly contemplated to be used both alone and in combination with other tests and indicia, whether quantitative or qualitative in nature, as described herein. The disclosed methods may be performed outside the body of a subject (ex vivo, for example in vitro).

Having described the invention in detail, it will be apparent that modifications, variations, and equivalent aspects are possible without departing the scope of the invention defined in the appended claims. Furthermore, it should be appreciated that all examples in the present disclosure are provided as non-limiting examples.

### EXAMPLES

The following non-limiting examples are provided to further illustrate aspects of the invention disclosed herein.

### Example 1: OLFM4 is Elevated in the Urine of Patients with AKI and Sepsis

### Patient Enrollment and Urine Collection

Patients were initially enrolled in the “AKI in Children Expected by Renal angina and Urinary Biomarkers” (AKI-CHERUB, NCT01735162) study at Cincinnati Children's Hospital Medical Center (CCHMC, see Menon et al. Urinary biomarker incorporation into the renal angina index early in intensive care unit admission optimizes acute kidney injury prediction in critically ill children: a prospective cohort study. Nephrology, Dialysis, Transplantation 2016; 31:586-594). Briefly, this was a prospective observational study conducted from September 2012 to March 2014 in the CCHMC pediatric ICU. All children admitted from ages 3 months to 25 years with a urinary catheter and anticipated discharge >48 hours from pediatric ICU admission were enrolled, excluding patients with end-stage renal disease (ESRD) and immediately post-renal transplant. Urine samples were collected, centrifuged, and stored at −80° C. For the purpose of this study, day 1 urine samples were used for OLFM4 analysis. Samples were chosen based on AKI and sepsis status. Sepsis diagnoses were extracted from the electronic medical record and categorized as yes/no based on ICU admission diagnosis in the ICU note. Demographic and lab data were extracted from the medical record. Neutrophil gelatinase associated lipocalin (NGAL) values were analyzed for the CHERUB study. The original study procedures were in accordance with the ethical standard of the CCHMC Institutional Review Board and in accordance with the Helsinki Declaration.

### AKI Staging

AKI staging was determined per the original AKI-CHERUB study protocol. Patients with AKI met criteria for severe, persistent AKI by Kidney Disease Improving Global Outcomes (KDIGO) serum creatinine criteria, or a >2 times change in serum creatinine from baseline present on day 3 of pediatric ICU admission. See Kellum JA, Mythen MG, Shaw AD. The 12th consensus conference of the Acute Dialysis Quality Initiative (ADQI XII). British J. Anaesthesia 2014; 113: 729-731.

### Urinary OLFM4 Measurement

A kit specific for human OLFM4 was developed by EMD Millipore (Burlington, MA). Urine OLFM4 concentration was measured following the kit protocol and assayed on a Luminex 200 Instrument (Austin, Texas). Briefly, OLFM4 was detected using an anti-OLFM4 antibody in a fluorescent bead-based immunoassay.

### Immunohistochemistry and Immunofluorescence

Human kidney block sections were selected by a pathologist who specializes in pediatric kidney disease. Four control sections were chosen from patients with Wilms' tumor or pathology not expected to have injury to the tubules, acknowledging that biopsies are not performed on pediatric patients with healthy kidneys. Four sections with acute tubular necrosis (ATN), three sections from patients with a renal transplant and tubular injury, and two sections from patients with chronic/ongoing tubular injury were prepared. Slides were deparaffinized and rehydrated with successive xylene, ethanol, and aqueous baths. Antigen retrieval was done by heating in citrate buffer. Endogenous biotin was blocked with Avidin biotin blocking kit (Biocare Medical, Concord, CA) and peroxidase blocked with Bloxall (Vector Laboratories, San Francisco, CA). Slides were then stained with anti-human OLFM4-PA5-85041 and Uromodulin-PA5-46959 (Thermo Scientific. Waltham, MA) followed by secondary antibodies. Images were taken on a Nikon TiE SpectraX microscope.

### Statistics

All statistical analyses were performed using SigmaStat (Systat Software, San Jose, CA) and GraphPad-Prism (San Diego, CA). As all groups were non-normally distributed, comparisons between different groups were performed using the Mann-Whitney U test, Kruskal Wallis test, and Spearman correlation. An area under the receiver operating curve (AUC-ROC) analysis was performed to assess whether OLFM4 could have discriminatory ability to differentiate patients with AKI from those without AKI. A cutoff p-value of 0.05 was used to establish statistical significance.

### RESULTS

### Patient Demographics

Urine from 8 patients without sepsis or AKI, 7 without sepsis but with AKI, 10 with sepsis but without AKI, and 11 with sepsis and AKI were analyzed (Table 2). The median age in each group ranged from 4.1-12.3 years, 50-90% of each group was female except the no sepsis/AKI group that had only 1 female, and the majority of patients were of white ethnicity. Neither median mechanical ventilation days, median ICU days, nor mean baseline serum creatinine levels differed between groups. The only patient who died had sepsis but no AKI, and 1 patient in each AKI group required renal replacement therapy (RRT).

### Patients with AKI Had Elevated Urine OLFM4 and NGAL Compared to Those Without AKI

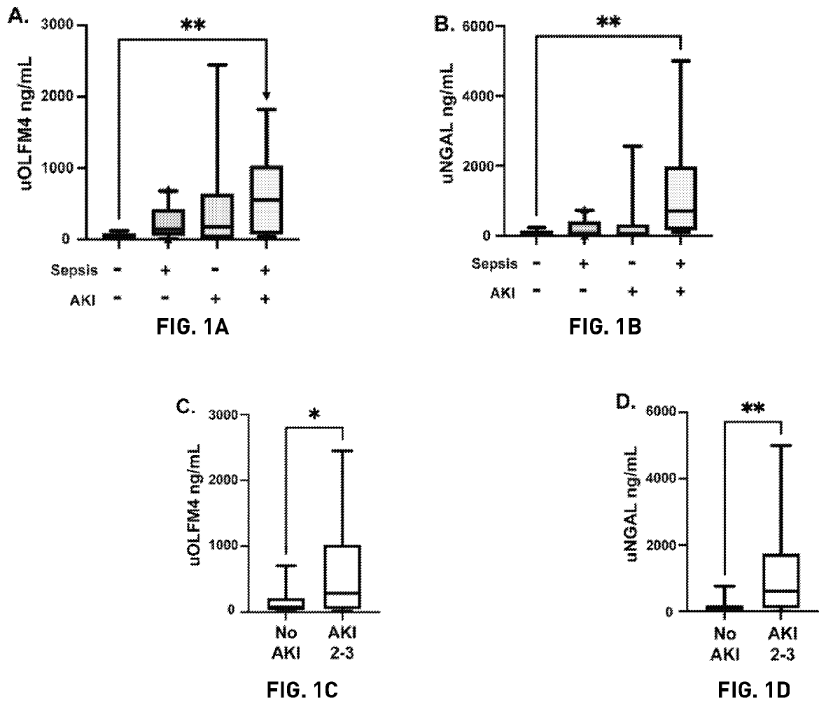

uOLFM4 concentration was assessed in all four groups of patients with and without sepsis and AKI (FIG. 1A). There was a trend toward higher uOLFM4 in each group with AKI, but this only reached statistical significance when comparing patients without sepsis or AKI to those with septic AKI (p=0.028). We performed the same analysis with urine NGAL, which produced similar results (FIG. 1B). Patients with septic AKI had higher NGAL concentrations than patients without sepsis or AKI (p=0.007).

We analyzed uOLFM4 from all 18 patients with AKI and compared them to the 18 patients with stage 1/no AKI. Patients with AKI had higher uOLFM4 (median 288.4 [IQR 48.9-1023] versus 81.7 ng/ml [IQR 27.66-211.4], p=0.044, FIG. 1C). These results are similar to urine NGAL levels seen in those with AKI compared to those without (n=18 and 17, respectively, median 612.4 [IQR 109-1738] versus 50.8 ng/mL [IQR 4.1-185.6], p=0.003, FIG. 1D).

### Patients with Sepsis Had Elevated Urine OLFM4 and Urine NGAL

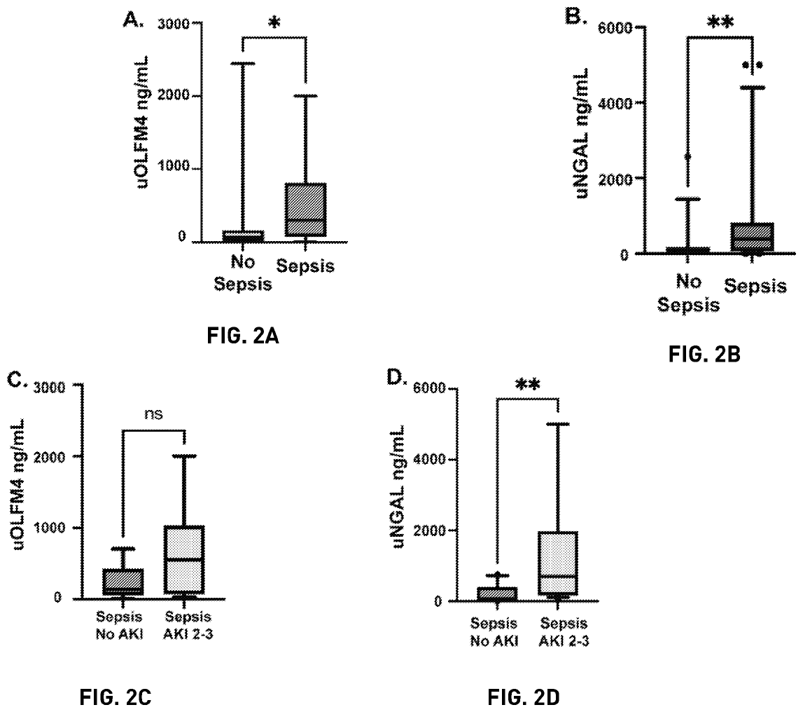

We compared urine from 21 patients with sepsis to 15 without sepsis. uOLFM4 was elevated in septic patients (median 302.1 [IQR 73.8-811.4] vs 59.06 ng/mL [IQR 21.84-178], p=0.026, FIG. 2A), as was urine NGAL (n=21 and 14, respectively, median 394.5 [IQR 69.4-808.3] vs 47.79 ng/mL [IQR 0.32-156.3], p=0.0095, FIG. 2B). Thus, both protein concentrations are elevated in the urine during sepsis.

To further assess the effect of sepsis and AKI on these urinary proteins, we compared just those patients with sepsis. uOLFM4 was not significantly higher in patients with septic AKI (n=11) compared to sepsis and no AKI (n=10, median 552.5 [IQR, 75.2-1030] versus 143.2 ng/mL [IQR 61.4-424.4], p=0.132, FIG. 2C). Urine NGAL was able to differentiate patients with septic AKI compared to sepsis and no AKI (median 701.6 [IQR 164.3-1978] vs 69.4 ng/mL [IQR 4.25-402.4], p=0.0027, FIG. 2D).

### Correlation Between uOLFM4 and NGAL

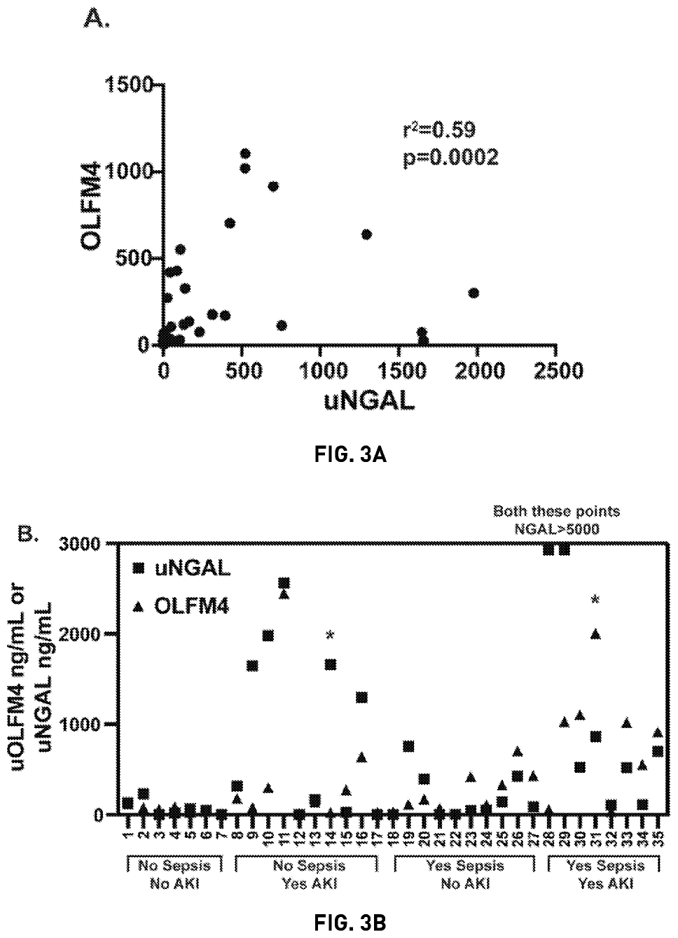

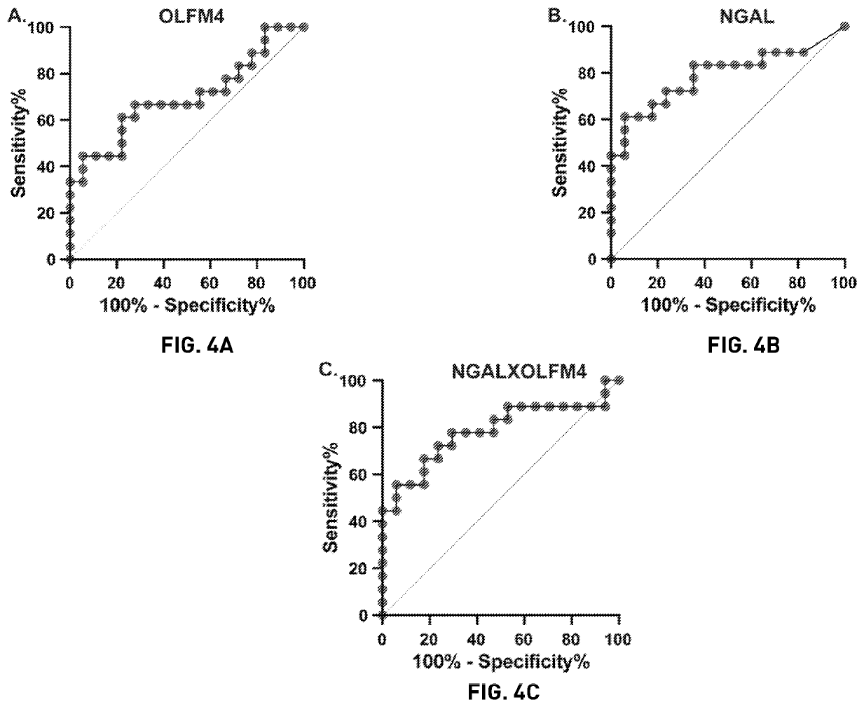

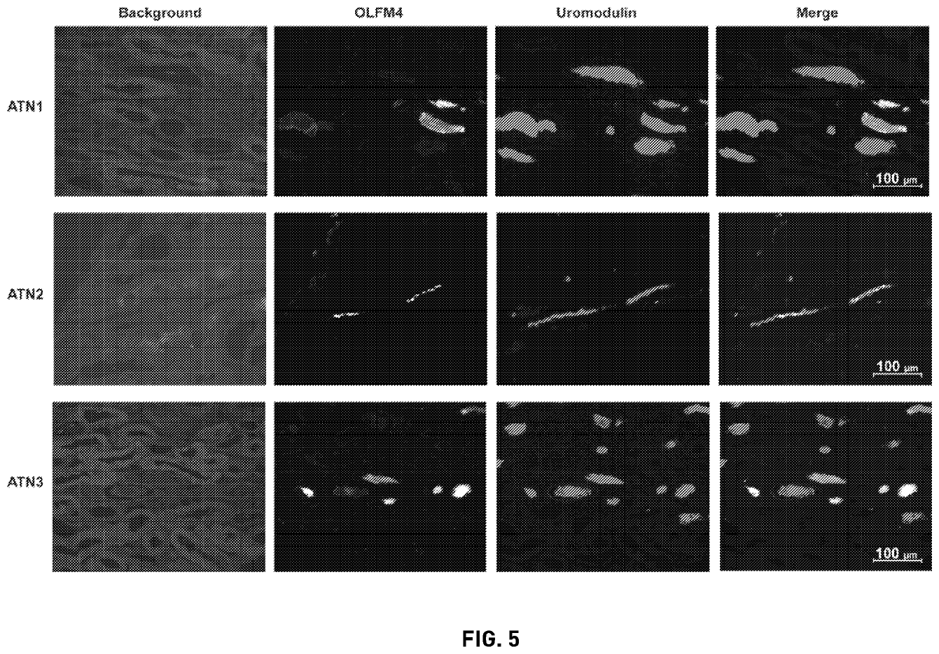

We observed correlation between urine NGAL and uOLFM4 (r2 0.59, 95% CI 0.304-0.773, p=0.002, FIG. 3A); however, there are some patients with high NGAL and low uOLFM4 and others with high uOLFM4 and low NGAL (FIG. 3B, asterisk). Finally, we compared the receiver operating curves for uOLFM4 and NGAL for predicting AKI. uOLFM4 had moderate ability to discriminate those with AKI from those without, with an AUC of 0.69 (95% CI, 0.52-0.87; FIG. 4A). NGAL had an AUC of 0.79 (95% CI, 0.63-0.94; FIG. 4B). We multiplied the two levels together to see if the product improved AUC for predicting kidney injury. The median of this product in patients with AKI was 196,988.2 [IQR 28,022.3-632, 124.1] versus 5,502.8 ng/mL [IQR 320.6-37,904.1] in those without AKI (p=0.0034). The AUC for the product of OLFM4 and NGAL for detecting stage 2-3 AKI was 0.78 (95% CI, 0.63-0.94; FIG. 4C), similar to that of NGAL alone (AUC 0.79, 95% CI, 0.63-0.94).

### OLFM4 Localizes to Human LOH

Two of four control samples had no detectable OLFM4 staining and two had rare OLFM4 expression, all of which colocalized to the LOH. From the nine samples with AKI, eight had moderate amounts of OLFM4 signal, almost all of which colocalized with uromodulin (FIG. 5). Of note, not all LOH cells produced OLFM4 as staining for OLFM4 was scattered throughout. In one patient with ATN of unknown etiology, OLFM4 did not always colocalize to LOH cells. Taken together, the vast majority of OLFM4 staining colocalized with uromodulin, suggesting OLFM4 expression comes from the LOH.

TABLE 2
Demographics and Outcomes by patient group.

| | No Sepsis | No Sepsis | Yes Sepsis | Yes Sepsis | |
|---|---|---|---|---|---|
| | No AKI | Yes AKI | No AKI | Yes AKI | |
| X | (n = 8) | (n = 7) | (n = 10) | (n = 11) | P value |
| Age, years, | 6.7 | 10.2 | 10.4 | 8.4 | 0.57 |
| mean (SD) | (7.3) | (6.6) | (5.6) | (4.9) | |
| Female (%) | 4 | 1 | 9 | 7 | |
| | (50) | (14) | (90) | (63) | |
| White | 6 | 5 | 9 | 10 | |
| Black | 1 | 2 | 1 | 0 | |
| Hispanic | 1 | 0 | 0 | 1 | |
| MV days, | 0.5 | 3 | 2 | 2 | 0.209 |
| median | (0.0-1.0) | (2.0-13.0) | (0.0-6.3) | (0.0-4.0) | |
| (IQR) | | | | | |
| ICU days, | 4.5 | 4 | 6.5 | 7 | 0.553 |
| median | (2.0-10.5) | (2.0-16.0) | (2.8-16.8) | (4.0-13.0) | |
| (IQR) | | | | | |
| Hospital | 11.5 | 8 | 26 | 11(8.0- | 0.621 |
| days, | (5.3-45.5) | (5.0-24.0) | (6.8-36.5) | 58.0) | |
| median | | | | | |
| (IQR) | | | | | |
| Need for | 0 | 0 | 1 | 1 | |
| RRT | | | | | |
| ICU | 0 | 0 | 1 | 0 | |
| Mortality | | | | | |
| Baseline | 0.43 | 0.51 | 0.44 | 0.37 | 0.473 |
| SCr, mean | (0.23) | (0.22) | (0.14) | (0.15) | |
| (SD) | | | | | |

MV—mechanical ventilation. ICU—intensive care unit. RRT—renal replacement therapy. SCr—serum creatinine.

### DISCUSSION

The clinical management and study of potential therapies for AKI have been hindered by imprecise, late, and inaccurate biomarkers used to diagnose this condition. There is a great deal of heterogeneity within patients with AKI that arises from injury to different regions of the nephron, different mechanisms of injury, and different genetic backgrounds. This example demonstrates that OLFM-4 protein is produced by human LOH cells and correlates with creatinine-diagnosed AKI, providing an LOH-specific AKI biomarker.

Tamm-Horsfall protein, or uromodulin, has been the main protein biomarker to date associated with the LOH. (Wen Y, Parikh CR. Current concepts and advances in biomarkers of acute kidney injury. Critical Reviews in Clinical Laboratory Sciences 2021; 58: 354-368; El-Achkar TM et al. Am J. Physiol. Renal Physiol. 2013; 304: F1066-1075). Previous work has shown a negative correlation between serum uromodulin and AKI in conditions like ANCA-associated vasculitis and ischemia-reperfusion injury (IRI), and between urine uromodulin and AKI in diabetes and patients undergoing cardiac surgery. Osteopontin, a bone phosphoprotein, is produced predominantly in the thick ascending limb of the LOH but also in the distal convoluted tubule. In states of injury, its expression is upregulated, which is why it has become a novel AKI biomarker. However, this upregulation is not tubule-segment specific and even occurs in the glomerulus. Chorley et al attempted to remedy the lack of nephron segment-specific biomarkers by using differential expression of urinary micro RNA (miRNA) to identify renal damage in a nephrotoxin-induced kidney injury model in rats. In this study, miR-221-3p, miR-222-3p, and miR-210-3p were increased in the urine of rats treated with thick ascending limb-specific nephrotoxic agents, suggesting these could be future LOH-specific biomarkers, but this technology has many steps prior to its implementation in clinical use. See Chorley BN, et al. Urinary miRNA Biomarkers of Drug-Induced Kidney Injury and Their Site Specificity Within the Nephron. Toxicological Sciences: An Official Journal of the Society of Toxicology 2021; 180: 1-16.

This example demonstrates a moderate correlation between OLFM4 and NGAL. Both are neutrophil granule proteins secreted by activated neutrophils and expressed by the nephron following injury. It remains difficult to know how much of these proteins in the urine came from glomerular filtering rather than tubular production. In bone marrow transplant experiments performed in septic mice, Stark et al found that mice null for OLFM4 in their blood neutrophils still produced OLFM4 locally by tubular cells. Stark et al. Am. J. Physiol. Renal Physiol. 2020; 318: F809-F816. Because of this and the patterns we observed in our immunofluorescence sections. we believe that the colocalization we observed is not related to leukocyte migration or selective filtration, but is reflective of de novo intrinsic kidney production. Tubular cells upregulate production of NGAL; but this production occurs non-specifically in the distal convoluted tubules, collecting ducts, and even to some extent the LOH, and there is reabsorption in the proximal tubules, limiting its anatomic specificity. See Singer E et al. Neutrophil gelatinase-associated lipocalin: pathophysiology and clinical applications. Acta Physiologica (Oxford, England) 2013; 207: 663-672.

Anatomic localization of biomarkers in the kidney is important given the unique roles each segment plays in renal function. The main roles of the LOH in clinical practice are its urinary concentrating abilities and pharmacologic manipulation with loop diuretics to induce natriuresis. In the past decade, the ability of tubular response to standardized doses of furosemide, known as the “furosemide stress test,” has shown ability to predict progression to advanced AKI stages (Chawla et al. supra) and receipt of renal replacement therapy (Lumlertgul et al. supra) better than new damage biomarkers (Koyner et al. supra). However, this test is only effective when the patient is intravascularly replete, and many providers don't feel comfortable giving furosemide when a patient is hemodynamically unstable. Therefore, the timing of furosemide administration is often when the patient has incurred significant and irreversible renal damage and is too late for predictive utility. A urinary biomarker that elevates early in the disease process and that predicts furosemide responsiveness would give clinicians a tool to predict who may benefit from RRT without having to wait for hemodynamic stability. Only 22 of 36 of patients in this study received furosemide, on average 2.8 days after PICU admission, and only 6 patients received 1 mg/kg IV or greater doses. Therefore, we were unable to assess whether uOLFM4 levels could predict a positive or negative furosemide stress test in this example. However, Example 2 below shows that uOLFM4 indeed can predict furosemide responsiveness and Example 3 provides further evidence of this.

Another application of OLFM4 as a biomarker is in the diagnosis of septic AKI. Sepsis is the most common etiology of AKI in the ICU, responsible for 40-50% of AKI in critically ill adults and children (34-36). Septic AKI is associated with increased fluid overload; greater oliguria; longer duration of mechanical ventilation, ICU, and hospital LOS (35-37). Additionally, septic AKI confers a 20-30% higher mortality in children than AKI of other etiologies (38,39). This etiology-specific AKI is especially difficult to diagnose. Increased fluid overload in this population dilutes serum creatinine, making this functional biomarker even less reliable. Additionally, less is known about biomarkers in this patient population. One of the main limitations of NGAL is its systemic elevation in inflammatory states, driven by IL-6 mediated hepatocyte production (40). An ideal marker of septic AKI would not be elevated by sepsis alone but would rise when a septic patient gets AKI. The data in this example show increased OLFM4 in patients with septic AKI. Having a septic AKI-specific biomarker would allow clinicians to target this particularly high-risk cohort and implement AKI mitigation protocols earlier.

In conclusion, OLFM4 is elevated in the urine of patients with AKI and sepsis, and there was a correlation between uOLFM4 and NGAL levels. Given OLFM4 colocalization to human LOH, these results indicate OLFM4 is a LOH-specific AKI biomarker. Example 2 below corroborates these findings prospectively, focusing on septic AKI, and evaluating whether OLFM4 can predict response to a standardized furosemide dose in patients with kidney injury. Example 3 provides further validation of OLFM4 as a biomarker for distinguishing AKI in both septic and non-septic patients, for predicting furosemide responsiveness, and for identifying patients most likely to be in need of RRT earlier in disease progression, before significant and irreversible renal damage has been incurred.

### Example 2: OLFM4 Predicts Furosemide Responsiveness

### Patient Enrollment and Urine Collection

Patient recruitment occurred as a convenience sampling from the larger TAKING FOCUS 2 study (IRB Number 2018-0724, PI Goldstein) ongoing at Cincinnati Children's Hospital Medical Center (CCHMC). Samples were collected between January 2020 and October 2021. Protocol details have been described previously (need reference). Namely, residual urine sent for a clinical or research neutrophil gelatinase associated lipocalin (NGAL) test from CCHMC's clinical laboratory were collected. Primarily urine samples from patients admitted the pediatric intensive care unit (ICU) were analyzed. We included multiple samples from a single patient when able, but only used one sample per patient per day. Demographic and lab data were extracted from the electronic medical record (EHR). Study protocols were in accordance with the ethical standard of the CCHMC Institutional Review Board and in accordance with the Helsinki Declaration.

### AKI Staging and Sepsis Determination

AKI was diagnosed using Kidney Disease Improving Global Outcomes (KDIGO) serum creatinine criteria. Severe, or a greater than 2 times change in serum creatinine from baseline, and persistent, present for greater than 48 hours, definitions were used, as these patients have been shown to have worse clinical outcomes. Highest serum creatinine on the sample date was used for classification of AKI status. Baseline creatinine was the lowest serum creatinine during that admission or in the 90 days prior to sample date. Attempts were made to try to account for severe volume overload and its impact on serum creatinine measurements in specific circumstances. Sepsis was determined using diagnoses pulled from the patients ICU progress note from the sample date. Specifically, if a patient had a positive culture (bacterial, viral, or fungal) or suspicion for infection, and hemodynamic changes or clinical worsening on or near the date of sample, the patient was considered to have sepsis. Patients with chronic colonization or lower titers of viruses that did not change the patient's clinical status were not considered to have sepsis.

### Urinary Biomarker Measurements

Urine from the patient sample was placed in a microcentrifuge tube, centrifuged per laboratory protocol, then the necessary amount of supernatant was removed for NGAL processing. The remaining urine was frozen at −80° C. Prior to analysis, samples were thawed and vortexed to resuspend proteins in the supernatant.

A custom kit specific for human OLFM4 was developed by EMD Millipore (Burlington, MA). Urine OLFM4 concentration was measured following the kit protocol and assayed on a Luminex 200 Instrument (Austin, Texas). Uromodulin was analyzed by enzyme linked immunosorbent assay (R&D Systems). NGAL levels were extracted from the EHR; of note, upper and lower limits of lab reporting are <50 and >18,000; for analysis, those with NGAL meeting those limits were recorded to have values of 50 ng/ml and 18,000 ng/ml, respectively. Similarly, the lower limit of detection for the uromodulin assay was 124 ng/ml and upper limit was 76000 ng/mL; these numbers were used for ratio calculation.

### Furosemide Responsiveness Testing

We collected furosemide administration time, dose, and the amount of urine (in milliliters) produced after the dose from the EHR. We used these values to calculate a urine flow rate (UFR), and greater than three mL per kilogram per hour of urine in the first four hours after furosemide dose was considered furosemide responsive. On rare occasions where a foley was not in place, urine was collected up to six hours post-dose. Only doses of 0.95 mg/kg or greater, administered zero to four days from OLFM4 sample date, were included in the analysis. When multiple doses were administered on the same day, the highest dose or first dose of the day was used.

### Statistics

All statistical analyses were performed using SAS version 9.4 (SAS Institute Inc., Cary, NC) and Graphpad Prism (San Diego, CA). As all groups were non-normally distributed, data are presented with median values followed by interquartile range (IQR). Pairwise comparisons were performed using Mann-Whitney U test, Kruskal Wallis test, and Spearman correlation on log transformed data. Linear mixed modeling was performed to account for lack of independence between samples when enough samples were available to account for samples coming from the same patient on different days. We calculated area under the receiver operating curve (AUC-ROC) for the ability of OLFM4 to predict furosemide responsiveness, and positive and negative predictive values were based on a Youden's index calculated to determine the OLFM4 level with the highest sensitivity and specificity for predicting furosemide responsiveness. A cutoff p-value of 0.05 was used to establish statistical significance.

### RESULTS

### Patient Cohort

65 patients contributed 178 unique urine samples. 33 samples represented no AKI and no sepsis, 15 no AKI and sepsis, 67 AKI but no sepsis, and 63 septic AKI. 130 of 178 samples (73%) were classified as AKI, while 78 of 178 (44%) came from patients with sepsis. The high percentage of AKI is likely due to samples being collected from residual NGAL assays, which are sent from patients with suspected AKI.

### uOLFM4 and uNGAL Elevated with AKI and Sepsis

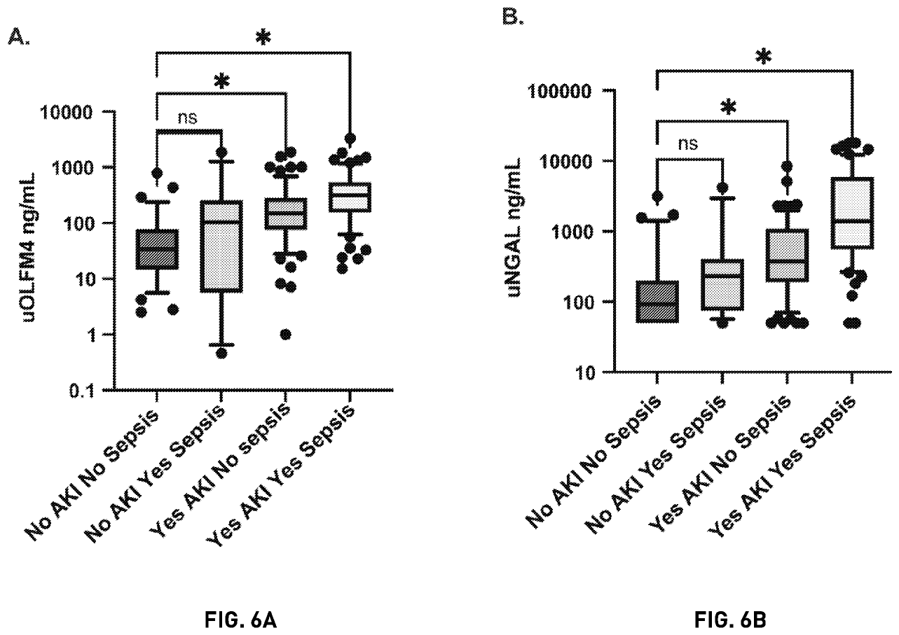

We first compared uOLFM4 and uNGAL concentrations between all four conditions with and without AKI and sepsis. Given the wide variation and lack of normal distribution for OLFM4 and NGAL, we log transformed measured values for all statistical calculations. OLFM4 levels were higher in patients with AKI, with (median 318 ng/ml [IQR 154-543]) and without sepsis (150 ng/mL [IQR 76-282]) when compared to patients without AKI or sepsis (34 ng/mL [IQR 15-78]) (FIG. 6A). We performed the same analysis with urine NGAL, which produced similar results (FIG. 6B). Both OLFM4 and NGAL were elevated in patients with AKI, regardless of sepsis status.

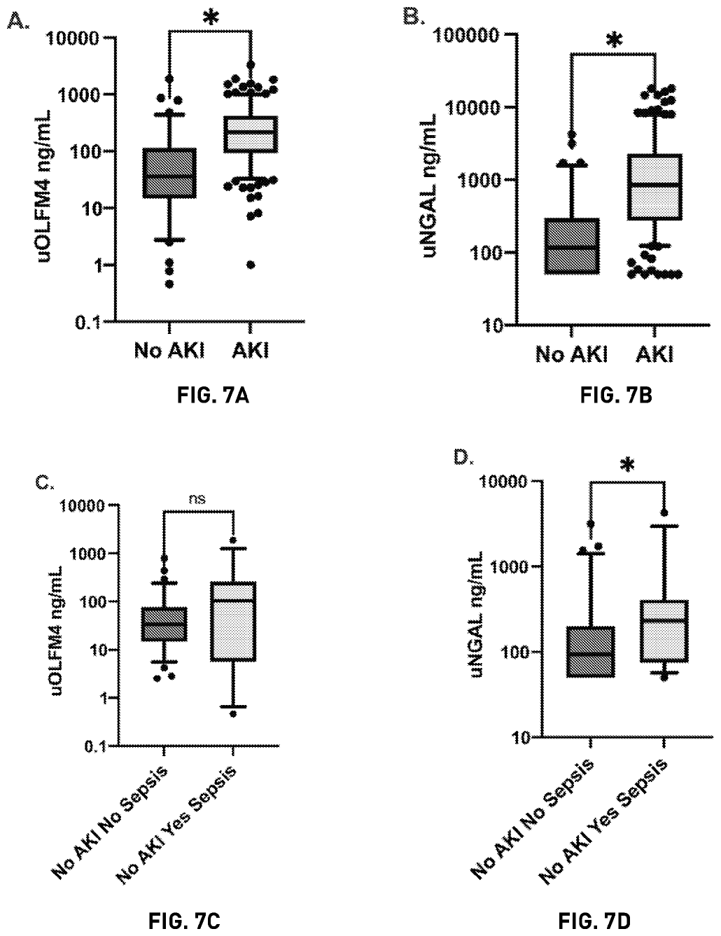

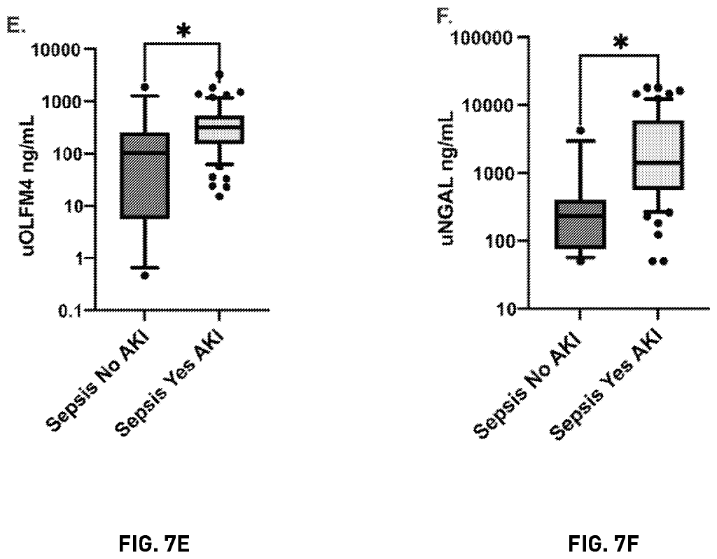

We next performed pairwise analyses looking at AKI and sepsis separately. When comparing uOLFM4 concentration was higher in samples with AKI (221 ng/mL [IQR 93-425]) compared to no AKI (36 ng/mL [IQR 15-115]; FIG. 7A). NGAL was also higher in patients with AKI (847 ng/mL [IQR 275-2304]) relative to those without AKI (115 ng/ml [IQR 50-295]; FIG. 7B). To test the effect of sepsis, we evaluated patients without AKI comparing those with and without sepsis. In this case, uOLFM4 was not different in those with sepsis compared to those with sepsis (FIG. 7C). In a similar comparison, NGAL was higher in those with sepsis (232 ng/mL [IQR 75-406]) compared to those without sepsis (93 ng/mL [IQR 50-201]; FIG. 7D). Finally, we tested in just those patients with sepsis, comparing those with AKI to those without AKI. In this case, both OLFM4 and NGAL were able to differentiate the two subgroups (FIG. 7E and FIG. 7F).

### uOLFM4 Predicted Furosemide Responsiveness

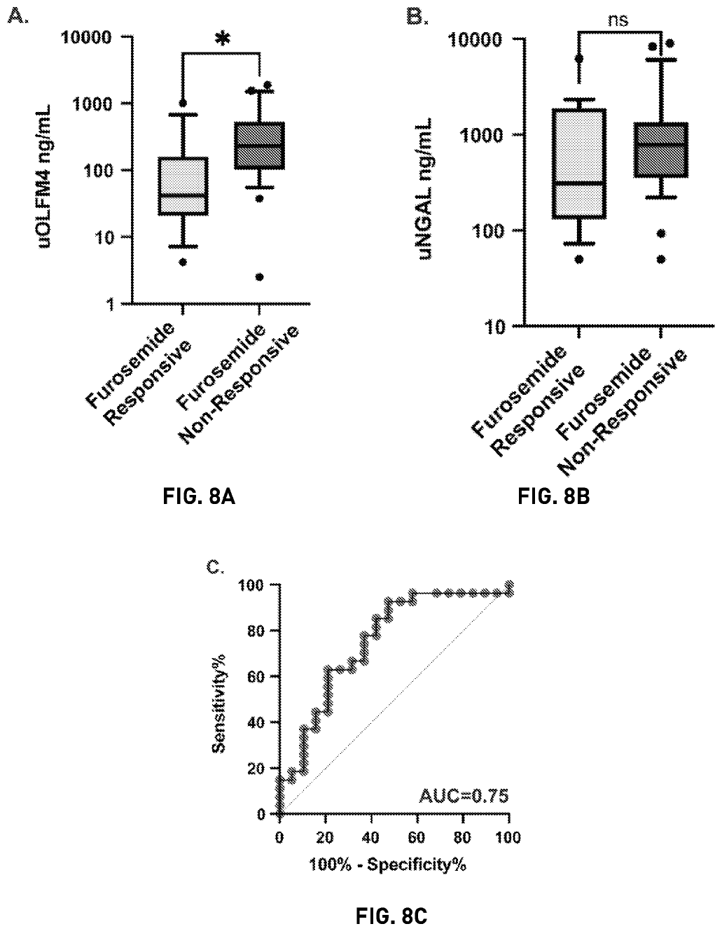

We next assessed uOLFM4 concentrations prior to furosemide administration. 55 uOLFM4 levels were available from 26 patients up to four days before 1 mg/kg of furosemide was given, with 48 of these samples (87%) within 48 hours of the furosemide dosing. The uOLFM4 concentration was lower in those who were furosemide responsive (42 ng/mL [IQR 21-161]) than in those who were not responsive (230 ng/mL [IQR 102-534]; FIG. 8A). This was not the case with uNGAL concentration, where sample concentrations were not different between those who responded to furosemide and those who did not (311[IQR 131-1902 ng/mL] vs 784 [IQR 354-1351 ng/mL], respectively; FIG. 8B). The median urine flow rate (UFR) in the furosemide responsive group was 4.4 mL/kg/hr (IQR 3.80-6.35) vs 1.44 mL/kg/hr (IQR 0.40-2.38) in the furosemide unresponsive group. There was also a negative correlation between OLFM4 and UFR (p=0.006) but not for NGAL and UFR (p=0.211). Finally, we assessed the receiver operating characteristic (ROC) curve for uOLFM predicting furosemide responsiveness. uOLFM4 had moderate ability to predict furosemide responsiveness with an area under the curve (AUC) of 0.751 (95% CI, 0.6018 to 0.8992, FIG. 8C). Using a uOLFM4 concentration cutoff of 50 ng/ml to predict a failure to respond to furosemide gives a positive predictive value of 89% and negative predictive value of 58%.

### Ratio of Uromodulin to uOLFM4 Improved AKI Diagnosis

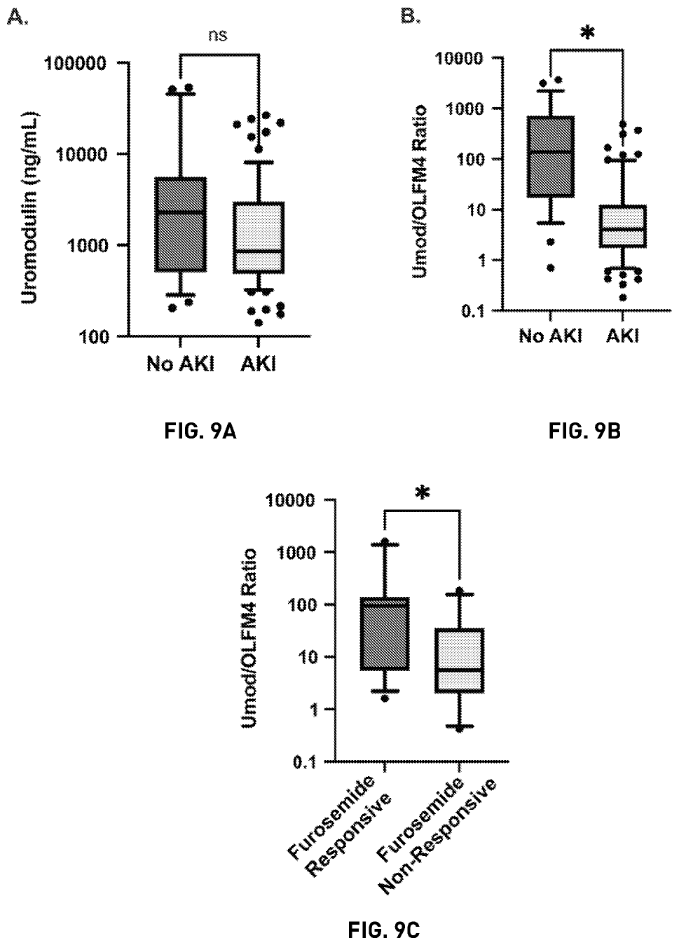

In a subset of samples, uromodulin concentrations were quantified to assess whether the ratio of uromodulin:OLFM4 could improve diagnostic accuracy of AKI. There were 101 samples analyzed, 25 in the no AKI group and 76 in the AKI group. Uromodulin alone in this smaller cohort was unable to differentiate AKI from no AKI. 854.0 ng/ml (IQR 483.5-2990) vs 2285 ng/ml (IQR 505.5-5623), p=0.16, (FIG. 9A). However, the uromodulin:OLFM4 ratio was able to differentiate AKI from no AKI, with lower values indicating AKI and higher values no AKI (4.0 [IQR 1.7-12.3] vs 136.7, [IQR 17.1-713.8]), p<0.0001, (FIG. 9B). The uromodulin:OLFM4 ratio also predicted to respond to furosemide with patients passing the test having a ratio of 94-3 [IRQ 5.5-140] compared to 5.6 [IRQ 2.0-35.4]; p=0.044) in those who did not, although this test was limited by the number of samples available (n=11 and 12, respectively; FIG. 9C).

### DISCUSSION

In this prospective observational study, the findings of Example 1 showing that uOLFM4 is capable of differentiating patients with AKI from those without are validated and extended to show a clinical utility of this biomarker in predicting response to furosemide.

This example validates the findings described in Example 1 that OLFM4 is increased in the urine of patients with AKI. The concentration of both NGAL and OLFM4 increase over 5-fold in the urine with kidney injury. Because both of these biomarkers are produced by neutrophils and secreted into plasma during sepsis, in addition to the nephron, the effect of sepsis alone in patients without AKI was evaluated. In these patients NGAL, but not OLFM4, increased in response to sepsis (FIG. 7C and FIG. 7D). Furthermore, in all patients with sepsis, both NGAL and OLFM4 differentiated between those patients with and without AKI (FIG. 7E and FIG. 7F). Thus serum OLFM4, produced and secreted by neutrophils in response to sepsis, does not preclude the utility of OLFM4 to diagnose kidney injury and the full contribution of serum OLFM4 to urinary levels will require further study.

As there is currently no therapy to treat AKI, emphasis is placed on early diagnosis, before injury becomes irreversible, and optimization of supportive care. See Vaidya VS, Ferguson MA, Bonventre JV. Biomarkers of acute kidney injury. Annual Review of Pharmacology and Toxicology 2008; 48: 463-493 (26). Current methods are unable to reliably predict AKI progression versus resolution. Lumlertgul et al proposed that the FST may better identify patients with high risk of AKI progression, and was able to show in a small pilot study that only 13% of FST responsive patients went on to receive RRT (Lumlertgul et al. Critical Care (London, England) 2018; 22: 101.). One of the major limitations of the FST is that a patient needs to be hemodynamically stable and euvolemic for this test to be administered. Given uOLFM4's ability to predict furosemide responsiveness without the need for hemodynamic stability, this biomarker may aid clinicians in determining which patients will go on to receive RRT.

There are few biomarkers available to identify injury to the LOH. Given that uromodulin is the best known LOH protein product, we sought to evaluate its ability to diagnose LOH injury and possibly augment OLFM4's diagnostic capability. Uromodulin is the most abundant urinary protein in physiologic conditions and serves multiple purposes, playing roles in renal ion transport, immunomodulation with antioxidant effects, and protecting against infection and nephrolithiasis. Uromodulin alone was not able to differentiate AKI from no AKI in a subset of this patient cohort (FIG. 9A). However, given injury to the LOH may disrupt production of uromodulin and OLFM4 production is increased with injury to the LOH, a uromodulin:OLFM4 ratio was evaluated to take advantage of both LOH proteins. As shown in FIG. 9B, a low ratio of uromodulin:OLFM4 did predict AKI and also, as shown in FIG. 9C, failure to respond to furosemide.

In conclusion, these results indicate that uOLFM4 can be used to identify patients with loop of Henle injury and predict furosemide responsiveness.

### Example 3 Urinary OLFM4 Predicts Acute Kidney Injury and Need for Renal Replacement Therapy

The patient cohort for Example 3 included PICU patients enrolled in TAKING FOCUS 2, Trial in AKI using NGAL and Fluid Overload to Optimize CRRT Use, who had a Renal angina index, RAI, calculated 12 hours after admission. RAI is a clinical scoring tool that uses patient characteristics and signs of renal injury, increase in creatinine or fluid overload, to stratify a patient's risk of developing AKI. If RAI was greater than 8, meaning the patient was at higher risk of developing AKI, an automated order is immediately placed for the nurse to collect a urine NGAL. If this NGAL level is >500, the providers in the PICU consider fluid restriction, or if the patient is already >10% fluid overloaded, consider RRT initiation. If the NGAL is between 150-500, the PICU provider will consider ordering a 1 mg/kg IV dose of furosemide, what we consider a furosemide stress test. The amount of urine output after an FST should help determine RRT need.

For this study, urine samples were collected from NGAL residuals or bladder catheter waste for up to 7 days. uOLFM4 levels were measured via Abcam enzyme linked immunosorbent assay and other clinical data were collected via chart review.

AKI was staged using KDIGO criteria, with severe AKI defined as a greater than two times increase in serum creatinine from baseline. Baseline creatinine was determined by the lowest level measured in the 3 months prior to admission or calculated with the modified Schwartz equation if prior creatinine was not available.

Between May 2022 and February 2023, 114 patients with RAI>8 were assessed for eligibility. 48 patients were excluded for lack of urine samples, due to anuria, undergoing bladder irrigations, or NGAL residuals disposed of in the lab. 66 patients had uOLFM4 measured and underwent chart review. 7 further patients were excluded for moderate or large leukocyte esterase present on urinalysis as OLFM4 is expressed in neutrophils so levels may be falsely elevated in patients with neutrophils present in their urine.

Ultimately, 59 patients were included. The first available urine sample was used for each patient and patients were grouped by their peak AKI stage or receipt of RRT within a 7-day study window. Of the 59 patients included, the median age was 11.6 with the majority being non-Hispanic white males. This was true across all groups. The majority of patients had pre-renal AKI but a number of patients had multifactorial AKI. Forty percent of patients met criteria for sepsis and two patients had prior diagnosis of CKD.

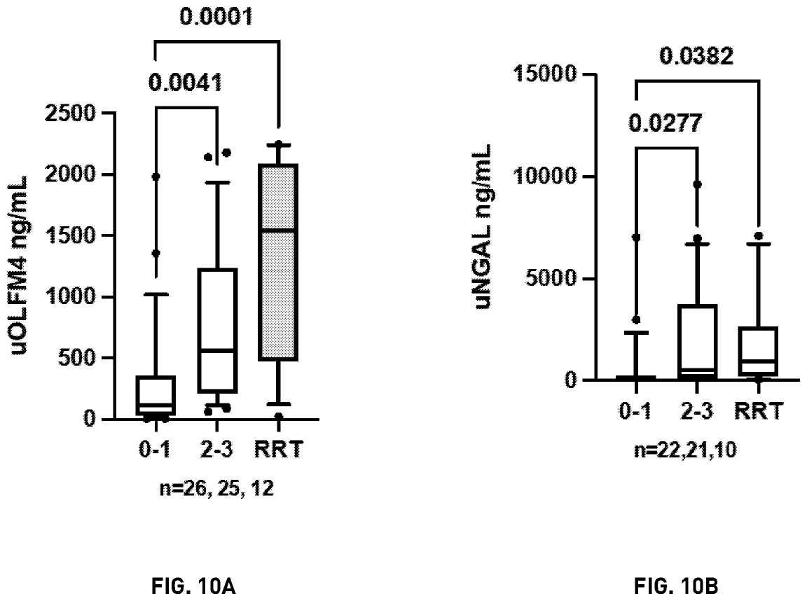

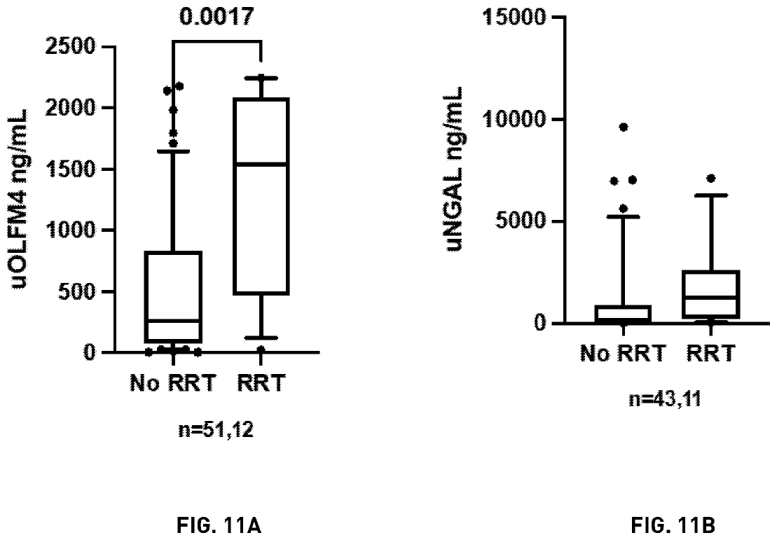

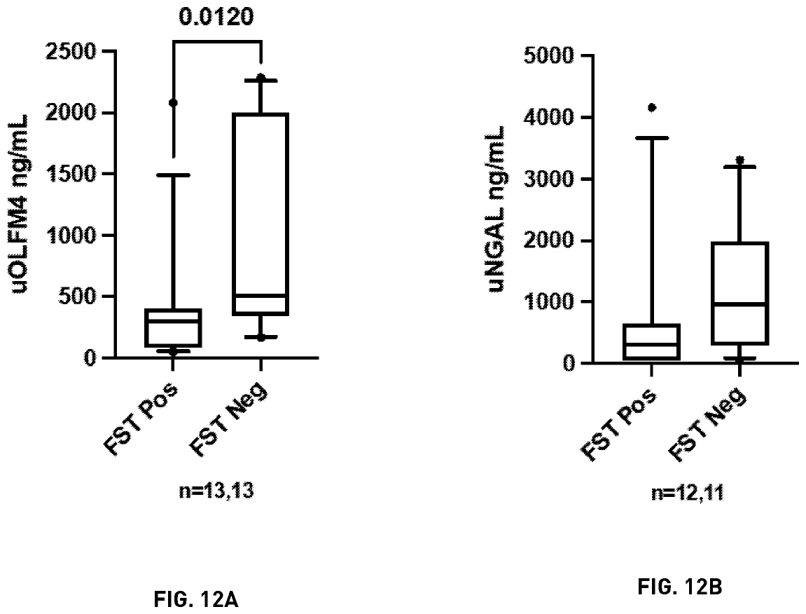

The results shown in FIG. 10A demonstrate that uOLFM4 levels are statistically significantly higher both in patients with severe AKI (defined as KIDGO stage 2-3) and in those who received RRT. FIG. 10B shows uNGAL levels in the same patient groups. FIG. 11A shows that uOLFM4 levels are statistically significantly higher in patients who received RRT compared to those who did not. FIG. 11B shows uNGAL levels in the same patient groups. FIG. 12A shows that uOLFM4 is elevated in patients who fail to respond to furosemide. FIG. 12B shows uNGAL levels in the same patient groups. Both patients with severe AKI and those who received RRT had statistically significantly higher uOLFM4 levels.

In summary, this cohort further validates the use of uOLFM4 to discriminate between patients with severe AKI from those with less severe forms and to identify patients who are likely to be furosemide unresponsive and who may benefit from RRT.

While the invention herein disclosed has been described by means of various aspects, specific embodiments and applications thereof, numerous modifications and variations could be made thereto by those skilled in the art without departing from the scope of the invention set forth in the claims.

It will be appreciated that the present invention is set forth in various levels of detail in this application. In certain instances, details that are not necessary for one of ordinary skill in the art to understand the invention, or that render other details difficult to perceive may have been omitted. The terminology used herein is for the purpose of describing particular embodiments only and is not intended to be limiting beyond the scope of the appended claims. Unless defined otherwise, technical terms used herein are to be understood as commonly understood by one of ordinary skill in the art to which the disclosure belongs.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein.

In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality.

Representative features are set out in the following clauses, which stand alone or may be combined, in any combination, with one or more features disclosed in the text and/or drawings of the specification.

The present disclosure provides a method for treating acute kidney injury (AKI) in a human subject in need thereof, the method comprising determining the amount of olfactomedin 4 (OLFM4) in a biological sample of the subject and administering renal replacement therapy (RRT) to the subject having an OLFM4 level above a pre-determined threshold value or administering supportive care to the subject having an OLFM4 level below the pre-determined threshold value.

The disclosure also provides a method for determining whether a human subject in need thereof is likely to be responsive or unresponsive to furosemide administration, the method comprising determining the amount of olfactomedin 4 (OLFM4) in a biological sample of the subject, wherein an OLFM4 level above a pre-determined threshold value indicates the subject is likely to be unresponsive and an OLFM4 level below the pre-determined threshold value indicates the subject is likely be furosemide responsive.

Furthermore, the disclosure provides a method for determining whether a human subject in need thereof is likely to progress to severe AKI, the method comprising determining the amount of olfactomedin 4 (OLFM4) in a biological sample of the subject, wherein an OLFM4 level above a pre-determined threshold value indicates disease progression is likely and an OLFM4 level below the pre-determined threshold value indicates disease progression is less likely.

In the present disclosure, the subject in need may be one diagnosed with stage 0-1 or stage 2-3 AKI, optionally using the Kidney Disease: Improving Global Outcomes (KDIGO) scale.

In the present disclosure, the subject in need may be one predicted to have kidney injury based on analysis of the subject's electronic medical record and/or one having urinary NGAL levels greater than 100 ng/ml, greater than 150 ng/ml, or greater than 200 ng/ml.

In the present disclosure, the subject in need may be hemodynamically unstable and/or wherein the subject is hypervolemic or hypovolemic.

In the present disclosure, the subject may not have been administered furosemide or received a furosemide stress test (FST) prior to determining the amount of OLFM4 in the biological sample.

According to the present disclosure, the biological sample may be a urine sample.

The supportive care may comprise one or more of fluid management, maintenance of euvolemia, prevention of hypotension, and avoidance of nephrotoxins.

The method of the disclosure may further comprise monitoring serum creatinine and urine output, optionally wherein the method further comprising detecting serum creatinine levels in one or more additional biological samples of the patient obtained at times following the initial determination of OLFM4.

The method may further comprise assaying serum and/or urine neutrophil gelatinase-associated lipocalin (NGAL) levels in a biological sample of the patient.

According to the disclosure, the pre-determined threshold value of OLFM4 may be 300 ng/ml, 350 ng/ml, 400 ng/ml, 450 ng/ml, 500 ng/ml, or 550 ng/ml in urine.

The method may further comprise determining an amount of uromodulin in a biological sample of the subject and calculating a ratio of uromodulin to OLFM4, wherein a ratio below a pre-determined threshold value indicates the subject is likely to progress to a more severe form of AKI, require RRT, and/or fail to respond to furosemide.

The ratio may be from 0.1-4, or wherein the ratio is selected from 4, 2, 1, 0.5, 0.25, and 0.1.

According to the present disclosure the subject may be diagnosed with sepsis.

The step of determining the amount of olfactomedin 4 (OLFM4) in the biological sample may comprise subjecting a portion of the sample to an immunoassay utilizing an anti-OLFM4 antibody.

The present disclosure also provides the use of urinary olfactomedin 4 (OLFM4) in an in vitro diagnostic method for identifying acute kidney injury (AKI) in a human subject, the method comprising determining an amount of olfactomedin 4 (OLFM4) in a urine sample of the subject.

The present disclosure also provides the use of urinary olfactomedin 4 (OLFM4) in an in vitro diagnostic method for predicting furosemide responsiveness in a human subject, the method comprising determining an amount of olfactomedin 4 (OLFM4) in a urine sample of the subject.

The present disclosure also provides the use of urinary olfactomedin 4 (OLFM4) in an in vitro diagnostic method for identifying a subject in need of renal replacement therapy (RRT), the method comprising determining an amount of olfactomedin 4 (OLFM4) in a urine sample of the subject.

The method may further comprise the step of comparing a determined amount of olfactomedin 4 (OLFM4) in the urine sample of the subject with a pre-determined threshold value, wherein an OLFM4 level above the pre-determined threshold value indicates the subject is likely to be suffering from AKI and an OLFM4 level below the pre-determined threshold value indicates the subject is less likely to be suffering from AKI.

The method may further comprise the step of comparing a determined amount of olfactomedin 4 (OLFM4) in the urine sample of the subject with a pre-determined threshold value, wherein an OLFM4 level above the pre-determined threshold value indicates the subject is likely to be furosemide unresponsive and an OLFM4 level below the pre-determined threshold value indicates the subject is likely be furosemide responsive; or

The method may further comprise the step of comparing a determined amount of olfactomedin 4 (OLFM4) in the urine sample of the subject with a pre-determined threshold value, wherein an OLFM4 level above a pre-determined threshold value indicates the subject is likely in need of renal replacement therapy (RRT) and an OLFM4 level below the pre-determined threshold value indicates the subject is less likely in need of renal replacement therapy (RRT).

The method may further comprise determining an amount of neutrophil gelatinase-associated lipocalin (NGAL) in a urine sample or in a serum sample of the subject.

The present disclosure also provides the use of olfactomedin 4 (OLFM4) as a biomarker for diagnosing acute kidney injury (AKI) in a human subject.

The OLFM4 may be urinary OLFM4 (uOLFM4).

1. A method for treating acute kidney injury (AKI) in a human subject in need thereof, the method comprising determining an amount of olfactomedin 4 (OLFM4) in a biological sample of the subject and administering renal replacement therapy (RRT) to the subject having an OLFM4 level above a pre-determined threshold value or administering supportive care to the subject having an OLFM4 level below the pre-determined threshold value.

2. A method for determining whether a human subject in need thereof is likely to be responsive or unresponsive to furosemide administration, the method comprising determining an amount of olfactomedin 4 (OLFM4) in a biological sample of the subject, wherein an OLFM4 level above a pre-determined threshold value indicates the subject is likely to be unresponsive and an OLFM4 level below the pre-determined threshold value indicates the subject is likely be furosemide responsive.

3. A method for determining whether a human subject in need thereof is likely to progress to severe AKI, the method comprising determining an amount of olfactomedin 4 (OLFM4) in a biological sample of the subject, wherein an OLFM4 level above a pre-determined threshold value indicates disease progression is likely and an OLFM4 level below the pre-determined threshold value indicates disease progression is less likely.

4. The method of claim 1, wherein the subject in need is one diagnosed with stage 0-1 or stage 2-3 AKI, optionally using the Kidney Disease: Improving Global Outcomes (KDIGO) scale.

5. The method of claim 1, wherein the subject in need is one predicted to have kidney injury based on analysis of the subject's electronic medical record and/or one having urinary NGAL levels greater than 100 ng/ml, greater than 150 ng/ml, or greater than 200 ng/ml.

6. The method of claim 1, wherein the subject is hemodynamically unstable and/or wherein the subject is hypervolemic or hypovolemic.

7. The method of claim 1, wherein the subject has not been administered furosemide or received a furosemide stress test (FST) prior to determining the amount of OLFM4 in the biological sample.

8. The method of claim 1, wherein the biological sample is a urine sample.

9. The method of claim 1, wherein supportive care comprises one or more of fluid management, maintenance of euvolemia, prevention of hypotension, and avoidance of nephrotoxins.

10. The method of claim 1, wherein the method further comprises monitoring serum creatinine and urine output, optionally wherein the method further comprising detecting serum creatinine levels in one or more additional biological samples of the patient obtained at times following the initial determination of OLFM4.

11. The method of claim 1, wherein the method further comprises assaying serum and/or urine neutrophil gelatinase-associated lipocalin (NGAL) levels in a biological sample of the patient.

12. The method of claim 1, wherein the pre-determined threshold value of OLFM4 is 300 ng/ml, 350 ng/ml, 400 ng/ml, 450 ng/ml, 500 ng/ml, or 550 ng/ml in urine.

13. The method of claim 1, wherein the method further comprises determining an amount of uromodulin in a biological sample of the subject and calculating a ratio of uromodulin to OLFM4, wherein a ratio below a pre-determined threshold value indicates the subject is likely to progress to a more severe form of AKI, require RRT, and/or fail to respond to furosemide.

14. The method of claim 13, wherein the ratio is from 0.1-4, or wherein the ratio is selected from 4, 2, 1, 0.5, 0.25, and 0.1.

15. The method of claim 1, wherein the subject is diagnosed with sepsis.

16. The method of claim 1, wherein determining the amount of olfactomedin 4 (OLFM4) in the biological sample comprises subjecting a portion of the sample to an immunoassay utilizing an anti-OLFM4 antibody.

17-27. (canceled)
